(19) United States
(12) Patent Application Publication
(10) Pub. No.: US 2021/0277145 A1
(43) Pub. Date: Sep. 9, 2021

(54) METHODS AND PHARMACEUTICAL COMPOSITIONS FOR INCREASING ENDOGENOUS PROTEIN LEVEL

(71) Applicants: INSERM (INSTITUT NATIONAL DE LA SANTE ET DE LA RECHERCHE MEDICALE), Paris (FR); UNIVERSITE PARIS-SUD, Orsay (FR)

(72) Inventors: Peter LENTING, Kremlin-Bicetre (FR); Gabriel AYME, Le Kremlin-Bicetre (FR); Olivier CHRISTOPHE, Le Kremlin-Bicetre (FR); Charlotte KAWECKI, Le Kremlin-Bicetre (FR); Cecile DENIS, Le Kremlin-Bicetre (FR)

(21) Appl. No.: 16/461,634

(22) PCT Filed: Nov. 16, 2017
(86) PCT No.: PCT/EP2017/079523

§ 371 Date: May 16, 2019

(30) Foreign Application Priority Data
Nov. 17, 2016 (EP) 16306502.2

(51) Int. Cl. C07K 16/38; C07K 16/36

(52) U.S. Cl. CPC C07K 16/38; C07K 2317/569; C07K 2317/31; C07K 16/36

(57) ABSTRACT

The present invention relates to a peptide comprising the amino acid sequence QGLIGDIALPRWGALWGDSV (SEQ ID NO: 1). Inventors have tested in wild-type mice a single domain antibody directed against VWF and tagged with an albumin-binding peptide. After giving a single dose intravenously (50 microgram/mouse), VWF levels were increased 8-15 fold for at least 7 days, knowing that the half-life of VWF is about 2-3 hours in a mouse. Moreover, intravenous administration of VWF together with a sdAb fused to an albumin-binding peptide resulted in detectable levels of VWF at 48 and 72 hours after injection, whereas no VWF could be detected when injected in the absence of such sdAb fused to an albumin-binding peptide. Thus, these results show a very long-lasting effect of this new approach.

### FIELD OF THE INVENTION

The invention relates to the use of a peptide to increase the half-life and the level of an endogenous protein.

### BACKGROUND OF THE INVENTION

In certain diseases a natural or endogenous protein is defective or missing in the patient, in particular because of inherited gene defects. In other diseases, the level of the natural or endogenous protein is not enough to have a normal function in the patient compared to a healthy person, the low of said endogenous protein is lower in the patient than in a healthy subject. There are many methods to overcoming these problems. Particularly, the use of polypeptides such as proteins for therapeutic applications has expanded in recent years mainly due to advanced knowledge of the molecular biological principles underlying many diseases and the availability of improved recombinant expression and delivery systems for human polypeptides. In the prior art, the short circulating half-life of polypeptide therapeutics has been addressed by covalent attachment of a polymer to the polypeptide. However, a number of problems have been observed with the attachment of polymers. For example, the attachment of polymers can lead to decreased drug activity. Furthermore, certain reagents used for coupling polymers to a protein are insufficiently reactive and therefore require long reaction times during which protein denaturation and/or inactivation can occur. Also, incomplete or non-uniform attachment leads to a mixed population of compounds having differing properties.

However there are not any methods in the art to increase the half-life and the level of endogenous proteins which are defective, missing or not enough to function correctly.

Thus, there is still a need to develop new products that increase the half-life and the level of the endogenous proteins to increase efficiency or reduce the amount of therapeutic proteins and/or frequency of infusions applied to patient. This would also reduce the costs of the treatment.

### SUMMARY OF THE INVENTION

The present invention relates to a peptide comprising the amino acid sequence QGLIGDIALPRWGALWGDSV (SEQ ID NO: 1). In particular, the invention is defined by the claims.

### DETAILED DESCRIPTION OF THE INVENTION

Inventors have tested in wild-type mice a single domain antibody directed against VWF and tagged with an albumin-binding peptide. After giving a single dose intravenously (50 microgram/mouse), VWF levels were increased 8-15 fold for at least 7 days, knowing that the half-life of VWF is about 2-3 hours in a mouse. Moreover, intravenous administration of VWF together with a sdAb fused to an albumin-binding peptide resulted in detectable levels of VWF at 48 and 72 hours after injection, whereas no VWF could be detected when injected in the absence of such sdAb fused to an albumin-binding peptide. Thus, these results show a very long-lasting effect of this new approach.

### Peptide of the Invention

Accordingly, in a first aspect, the invention relates to a peptide comprising the amino acid sequence QGLIGDIALPRWGALWGDSV (SEQ ID NO: 1).

In one embodiment, the peptide of the invention consists in the amino acid sequence as set forth in SEQ ID NO:1 comprising at least 75%, preferably at least 80%, 85%, 90%, 95%, 96%, 97%, 98%, 99%, 99.5% or 99.9% identity with SEQ ID NO:1.

In a particular embodiment, the peptide comprises or consists of an amino acid sequence

(SEQ ID NO: 1)
QGLIGDIALPRWGALWGDSV.

As used herein, the term “peptide” corresponds to the chemical agents belonging to the protein family. A peptide is composed of a mixture of several amino acids. Depending on the number of amino acids involved, peptides are categorized as dipeptides, composed of 2 amino acids, tripeptides, made up of 3 amino acids, and so on. Peptides composed of more than 10 amino acids are called polypeptides. Thus, the peptide of the invention can be considered as a polypeptide.

The peptides according to the invention, may be produced by conventional automated peptide synthesis methods or by recombinant expression. General principles for designing and making proteins are well known to those of skill in the art.

Peptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. Peptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. Nos. 6,569,645; 6,043,344; 6,074,849; and 6,579,520 provide specific examples for the recombinant production of peptides and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, W138, and the like have specific cellular machinery and characteristic mechanisms for such post-translational activities and may be chosen to ensure the correct modification and processing of the introduced, foreign protein.

In some embodiments, the invention relates to a nucleic acid encoding an amino acid sequence comprising SEQ ID NO: 1. Nucleic acids of the invention may be produced by any technique known per se in the art, such as, without limitation, any chemical, biological, genetic or enzymatic technique, either alone or in combination(s).

In another embodiment, the invention relates to an expression vector comprising a nucleic acid sequence encoding an amino sequence comprising SEQ ID NO: 1. According to the invention, expression vectors suitable for use in the invention may comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

In some embodiments, the invention relates to a host cell comprising the expression vector as descried above. Examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art. In another embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

### Drug Conjugates

The inventors have shown that the peptide as described above can be linked with a single domain antibody to increase the half-life and the level of an endogenous protein.

Accordingly, in a second aspect, the invention relates to a drug conjugate comprising the peptide according to the invention linked to a heterologous moiety.

In some embodiments, the heterologous moiety is an aptamer, a nucleic acid, another polypeptide or an isolated single domain antibody.

In some embodiments, the peptide of the present invention is conjugated to the heterologous moiety. As used herein, the term “conjugation” has its general meaning in the art and means a chemical conjugation. Techniques for conjugating heterologous moiety to polypeptides, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. In particular the one skilled in the art can also envisage a polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882). The term “transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g. a peptide-bound lysine, resulting in a ε-(γ-glutamyl) lysine isopeptide bond. TGases include, inter alia, bacterial transglutaminase (BTG) such as the enzyme having EC reference EC 2.3.2.13 (protein-glutamine-γ-glutamyltransferase). In some embodiments, the single domain antibody of the present invention is conjugated to the heterologous moiety by a linker molecule. As used herein, the term “linker molecule” refers to any molecule attached to the peptide of the present invention. The attachment is typically covalent. In some embodiments, the linker molecule is flexible and does not interfere with the binding of the peptide of the present invention.

In some embodiments, when the heterologous moiety is an isolated single domain antibody, the peptide of the present invention is fused to the isolated single domain antibody to form a fusion protein.

According to the invention, the fusion protein comprises an isolated single domain antibody (sbAb) that is fused either directly or via a spacer at its C-terminal end to the N-terminal end of the peptide, or at its N-terminal end to the C-terminal end of the peptide. As used herein, the term “directly” means that the (first or last) amino acid at the terminal end (N or C-terminal end) of the single domain antibody is fused to the (first or last) amino acid at the terminal end (N or C-terminal end) of peptide. In other words, in this embodiment, the last amino acid of the C-terminal end of said sdAb is directly linked by a covalent bond to the first amino acid of the N-terminal end of said peptide, or the first amino acid of the N-terminal end of said sdAb is directly linked by a covalent bond to the last amino acid of the C-terminal end of said peptide. As used herein, the term “spacer” also called “linker” refers to a sequence of at least one amino acid that links the sdAb to peptide of the invention. Such a spacer may be useful to prevent steric hindrances. Examples of linkers that could be used include, but are not limited to, have the following sequences (Gly3-Ser)4, (Gly3-Ser), Ser-Gly or (Ala-Ala-Ala).

As used herein, the term “single domain antibody” has its general meaning in the art and refers to the single heavy chain variable domain of antibodies of the type that can be found in Camelid mammals which are naturally devoid of light chains. Such single-domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al, Trends Biotechnol, 2003, 21(11):484-490; and WO 06/030220, WO 06/003388. The amino acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementary Determining Region 1” or “CDR1”; as “Complementarity Determining Region 2” or “CDR2” and as “Complementarity Determining Region 3” or “CDR3”, respectively. Accordingly, the single-domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single-domain antibody are numbered according to the general numbering for VH domains given by the International ImMunoGeneTics information system aminoacid numbering (http://imgt.cines.fr/).

By “isolated” it is meant, when referring to a single-domain antibody according to the invention, that the indicated molecule is present in the substantial absence of other biological macromolecules of the same type.

In a particular embodiment, the drug conjugate according to the invention, wherein the isolated single domain antibody is directed against at least one protein or a derivative thereof selected from the group consisting of: Von Willebrand factor, Fibrinogen, Factor II (prothrombin), Factor V, Factor VII, Factor VIII, Factor IX, Factor X, Factor XI, Factor XII, Factor XIII, Protein C, Protein S, Protein Z, Protein Z-inhibitor, Tissue factor pathway inhibitor (TFPI), α1-antitrypsin inhibitor, Thrombin Activatable Fibrinolysis Inhibitor (TAFI)/carboxypeptidase B2, Antithrombin, α2-antiplasmin, Plasmin Activator Inhibitor-1 (PAI-1), Plasminogen, tissue plasminogen activator (tPA), urinary plasminogen activator (uPA), ADAMTS13, Complement protein C2, Complement protein C3, Complement protein C4, Complement protein C6, Complement factor H, Complement factor I, Properdin, Ceruloplasmin, Kininogen, Thrombopoietin, Erytropoeitin, Soluble Mannose-binding lectin, Interferon-α, Interferon-β, Interferon-γ, Granulocyte colony-stimulating factor, Granulocyte-macrophage colony-stimulating factor, Keratinocyte growth factor, Interleukin-2, Interleukin-6, Interleukin-7, Interleukin-10, Interleukin-11, Interleukin-12, Interleukin-15, Interleukin-21, Growth hormone-releasing hormone and Hyaluronidase.

In a particular embodiment, the drug conjugate according to the invention, wherein the isolated single domain antibody is directed against the von Willebrand factor (VWF).

The term “VWF” has its general meaning in the art and refers to the human von Willebrand factor (VWF) which is a blood glycoprotein involved in blood clotting. VWF is a monomer composed of several homologous domains each covering different functions: D1-D2-D′-D3-A1-A2-A3-D4-C1-C2-C3-C4-C5-C6-CK. The naturally occurring human VWF gene has a nucleotide sequence as shown in Genbank Accession number NM_000552.4 and the naturally occurring human VWF protein has an aminoacid sequence as shown in Genbank Accession number NP_000543.2. The murine nucleotide and amino acid sequences have also been described (Genbank Accession numbers NM_011708.4 and NP_035838.3). Monomers are subsequently arranged into dimers or multimers by crosslinking of cysteine residues via disulfide bonds. Multimers of VWF can thus be extremely large and can consist of over 40 monomers also called high molecular weight (HMW)-multimers of VWF.

In a particular embodiment, the isolated single domain antibody is directed against DD′3 domain of VWF. Particularly, the isolated single-domain antibody directed against von VWF D′D3 domain does not induce the unfolding of VWF (which leads to exposure of platelet-binding sites). Moreover, within the context of the invention the single-domain antibody directed against von VWF D′D3 domain does not block the binding to VWF of a polypeptide such as a clotting factor comprising such single-domain antibody as described below.

In a particular embodiment, the isolated single domain antibodies directed against DD′3 are selected from the group consisting of: KB-VWF-013; KB-VWF-008 and KB-VWF-011.

The drug conjugate according to the invention, wherein the isolated single domain antibody comprises:

i) a CDR1 having least 70% of identity with sequence set forth as SEQ ID NO:2, a CDR2 having at least 70% of identity with sequence set forth as SEQ ID NO:3 and a CDR3 having at least 70% of identity with sequence set forth as SEQ ID NO:4;

ii) a CDR1 having least 70% of identity with sequence set forth as SEQ ID NO:7, a CDR2 having at least 70% of identity with sequence set forth as SEQ ID NO:8 and a CDR3 having at least 70% of identity with sequence set forth as SEQ ID NO:9;

iii) a CDR1 having least 70% of identity with sequence set forth as SEQ ID NO:12, a CDR2 having at least 70% of identity with sequence set forth as SEQ ID NO:13 and a CDR3 having at least 70% of identity with sequence set forth as SEQ ID NO:14;

In a particular embodiment, the drug conjugate according to the invention, wherein said isolated single domain antibody is fused to the peptide according to the invention comprising a sequence set forth as SEQ ID NO: 6; SEQ ID NO: 11 or SEQ ID NO: 16.

The sequences of KB-VWF-013 domains, KB-KB-VWF-008 domains, KB-KB-VWF-011 domains and their fusion with the peptide of the invention are indicated in the following table (A):

TABLE A

| KB-VWF-013 | |
|---|---|
| domains | Sequences |
| CDR1 | SEQ ID NO: 2 |
| | GRTFIRYAMA |
| CDR2 | SEQ ID NO: 3 |
| | IPQSGGRSYYADSVKG |
| CDR3 | SEQ ID NO: 4 |
| | TSTYYGRSAYSSHSGGYDY |
| SEQUENCE KB- | SEQ ID NO: 5 |
| VWF-013 | QVQLVQSGGGLVQAGDSLRLSCAASGRTFIRYAMAWFRQAPGKEREFVAAI |
| | PQSGGRSYYADSVKGRFTISRDNAKNTVYLQMNSLKPEDTAVYSCAATSTYY |
| | GRSAYSSHSGGYDYWGQGTQVTVSS |
| | (Bold: CDR sequence and not bold: framework sequence) |
| SEQUENCE | SEQ ID NO: 6 |
| KB-VWF-013bv- | QVQLQSGGGLVQAGDSLRLSCAASGRTFIRYAMAWFRQAPGKEREFVAAIP |
| ABP | QSGGRSYYADSVKGRFTISRDNAKNTVYLQMNSLKPEDTAVYSCAATSTYYG |
| | RSAYSSHSGGYDYWGQGTQVTVSSGGGSGGGSGGGSGGGSQVQLVQSG |
| | GGLVQAGDSLRLSCAASGRTFIRYAMAWFRQAPGKEREFVAAIPQSGGRSYY |
| | ADS VKGRFTISRDNAKNTVYLQMNSLKPEDTAVYSCAATSTYYGRSAYSSHS |
| | GGYDYWGQGTQVTVSSGRGGGSLTPRGVRLGGGSQGLIGDIALPRWGALW |
| | GDSV |
| | Italic: linker sequence ; Underlined: thrombin-cleavage |
| | site ; Bold: ABP (peptide) |
| KB-VWF-008 | |
| domains | Sequences |
| CDR1 | SEQ ID NO: 7 |
| | GRTFSDYAMG |
| CDR2 | SEQ ID NO: 8 |
| | INRSGGRLSYAESVND |
| CDR3 | SEQ ID NO: 9 |
| | RTNWNPPRPLPEEYNY |
| SEQUENCE KB- | SEQ ID NO: 10 |
| VWF-008 | QVQLVQSGGGLVQAGDSLKLSCAASGRTFSDYAMGCILQNPGKERDFVASIN |
| | RSGGRLSYAESVNDLFTISVDNAKNMLYLQMNSLKPEDTAVHYCVLRTNWNP |
| | PRPLPEEYNYWGQETQVTVSS |
| | (Bold: CDR sequence and not bold: framework sequence) |
| SEQUENCE | SEQ ID NO: 11 |
| KB-VWF-008bv- | QVQLVQSGGGLVQAGDSLKLSCAASGRTFSDYAMGCILQNPGKERDFVASIN |
| ABP | RSGGRLSYAESVNDLFTISVDNAKNMLYLQMNSLKPEDTAVHYCVLRTNWNP |
| | PRPLPEEYNYWGQETQVTVSSGGGSGGGSGGGSGGGSQVQLVQSGGGLV |
| | QAGDSLKLSCAASGRTFSDYAMGCILQNPGKERDFVASINRSGGRLSYAESV |
| | NDLFTISVDNAKNMLYLQMNSLKPEDTAVHYCVLRTNWNPPRPLPEEYNYWG |
| | QETQVTVSSGRGGGSLTPRGVRLGGGSQGLIGDIALPRWGALWGDSV |
| | Italic: linker sequence ; Underlined: thrombin-cleavage |
| | site ; Bold: ABP (peptide) |
| KB-VWF-011 | |
| domains | Sequences |
| CDR1 | SEQ ID NO: 12 |
| | GGTFSNYAMG |
| CDR2 | SEQ ID NO: 13 |
| | ISRSGHRTDYADSAKG |
| CDR3 | SEQ ID NO: 14 |
| | RSDWSIATTATSYDY |
| SEQUENCE KB- | SEQ ID NO: 15 |
| VWF-011 | QVQLVQSGGGLVQAGDSLRLSCAASGGTFSNYAMGWFRQTPGKEREFVARI |
| | SRSGHRTDYADSAKGRFTISRDNAKNTVYLQMNSLKPEDTAVYYCAARSDW |
| | SIATTATSYDYWGQGTQVTVSS |
| | (Bold: CDR sequence and not bold: framework sequence) |
| SEQUENCE | SEQ ID NO: 16 |
| KB-VWF-011bv- | QVQLVQSGGGLVQAGDSLRLSCAASGGTFSNYAMGWFRQTPGKEREFVARI |
| ABP | SRSGHRTDYADSAKGRFTISRDNAKNTVYLQMNSLKPEDTAVYYCAARSDW |
| | SIATTATSYDYWGQGTQVTVSSGGGSGGGSGGGSGGGSQVQLVQSGGGLV |
| | QAGDSLRLSCAASGGTFSNYAMGWFRQTPGKEREFVARISRSGHRTDYADS |
| | AKGRFTISRDNAKNTVYLQMNSLKPEDTAVYYCAARSDWSIATTATSYDYWG |
| | QGTQVTVSSGRGGGSLTPRGVRLGGGSQGLIGDIALPRWGALWGDSV |
| | Italic: linker sequence; Underlined: thrombin-cleavage |
| | site; Bold: ABP |

Amino acid sequence identity is preferably determined using a suitable sequence alignment algorithm and default parameters, such as BLAST P (Karlin and Altschul, Proc. Natl Acad. Sci. USA 87(6):2264-2268 (1990)).

In a particular embodiment, the drug conjugate according to the invention, wherein the isolated single domain antibody is directed against the Antithrombin (AT). As used herein, the term “antithrombin” or (AT) also known as antithrombin III (AT III) refers to an anticoagulant factor which prevents the coagulation of blood. It is considered as a serpin (serine protease inhibitor) and is thus similar in structure to most other plasma protease inhibitors, such as alpha 1-antichymotrypsin, alpha 2-antiplasmin and Heparin cofactor II. It inhibits thrombin, FXa and other serine proteases functioning in the coagulation pathway. It consists of 432 amino acids, is produced by the liver hepatocyte and has a long plasma half-life of two and half days (Collen, Schetz et al. 1977). The amino acid sequence of AT is well-conserved and the homology among cow, sheep, rabbit, mouse and human is 84%-89% (Olson and Bjork 1994). Although the primary physiological targets of AT are thrombin and FXa, AT also inhibits FIXa, FX1a, FXI1a, as well as FVIIa to a lesser extent. AT exerts its inhibition together with heparin. In presence of heparin the inhibition rate of thrombin and FXa by AT increases by 3 to 4 orders of magnitude from 7-11×10³ M⁻¹ s⁻¹ to 1.5-4×10⁷ M⁻¹ s⁻¹ and from 2.5×10³ M⁻¹ s⁻¹ to 1.25-2.5×10⁷ M⁻¹ s⁻¹, respectively (Olson, Swanson et al. 2004). Unlike TFPI and APC, which inhibit coagulation solely at the initiating stage and the amplification stage respectively, AT exerts its inhibition on coagulation at both the initiation and amplification stage.

In a particular embodiment, the isolated single domain antibodies directed against AT are selected from the group consisting of: KB-AT-001, KB-AT-002, KB-AT-003, KB-AT-004, KB-AT-005, KB-AT-006 and KB-AT-007.

The drug conjugate according to the invention, wherein the isolated single domain antibody comprises:

i) a CDR1 having least 70% of identity with sequence set forth as SEQ ID NO:17, a CDR2 having at least 70% of identity with sequence set forth as SEQ ID NO:18 and a CDR3 having at least 70% of identity with sequence set forth as SEQ ID NO:19;

ii) a CDR1 having least 70% of identity with sequence set forth as SEQ ID NO:21, a CDR2 having at least 70% of identity with sequence set forth as SEQ ID NO:22 and a CDR3 having at least 70% of identity with sequence set forth as SEQ ID NO:23;

iii) a CDR1 having least 70% of identity with sequence set forth as SEQ ID NO:25, a CDR2 having at least 70% of identity with sequence set forth as SEQ ID NO:26 and a CDR3 having at least 70% of identity with sequence set forth as SEQ ID NO:27;

iv) a CDR1 having least 70% of identity with sequence set forth as SEQ ID NO:29, a CDR2 having at least 70% of identity with sequence set forth as SEQ ID NO:30 and a CDR3 having at least 70% of identity with sequence set forth as SEQ ID NO:31;

v) a CDR1 having least 70% of identity with sequence set forth as SEQ ID NO:33, a CDR2 having at least 70% of identity with sequence set forth as SEQ ID NO:34 and a CDR3 having at least 70% of identity with sequence set forth as SEQ ID NO:35;

vi) a CDR1 having least 70% of identity with sequence set forth as SEQ ID NO:37, a CDR2 having at least 70% of identity with sequence set forth as SEQ ID NO:38 and a CDR3 having at least 70% of identity with sequence set forth as SEQ ID NO:39; or

vii) a CDR1 having least 70% of identity with sequence set forth as SEQ ID NO:41, a CDR2 having at least 70% of identity with sequence set forth as SEQ ID NO:42 and a CDR3 having at least 70% of identity with sequence set forth as SEQ ID NO:43;

The sequences of KB-AT-001, KB-AT-002, KB-AT-003, KB-AT-004, KB-AT-005, KB-AT-006 and KB-AT-007 are indicated in the following table (B):

TABLE B

| KB-AT-001 | |
|---|---|
| domains | Sequences |
| CDR1 | SEQ ID NO: 17 |
| | GRTFRNYV |
| CDR2 | SEQ ID NO: 18 |
| | INRSGAIT |
| CDR3 | SEQ ID NO: 19 |
| | AAGETTWSIRRDDYDY |
| SEQUENCE | SEQ ID NO: 20 |
| KB-AT-001 | QVQLQQSGGDLAQRGGSLRLSCAASGRTFRNYVMGWFRQAPGKDPEF |
| | IAGINRSGAITYYGDSVKGRFTISRDNAKNTVSLQMNSLEPEDTAVYYC |
| | AAGETTWSIRRDDYDYWGQGTQVTVSS |
| | (Bold: CDR sequence and not bold: framework sequence) |
| KB-AT-002 | |
| domains | Sequences |
| CDR1 | SEQ ID NO: 21 |
| | SGRTFNNNG |
| CDR2 | SEQ ID NO: 22 |
| | ISWSGGST |
| CDR3 | SEQ ID NO: 23 |
| | AARTRYNSGLFSRNYDY |
| SEQUENCE | SEQ ID NO: 24 |
| KB-AT-002 | QVQLVQSGGGLVQAGGSLRLSCAASGRTFNNNGMGWFRQAPGKERE |
| | FVAAISWSGGSTYYADSVKGRYIMSRDNAKNTVYLQMNSLKPEDTAV |
| | YYCAARTRYNSGLFSRNYDYWGQGTQVTVSS |
| | (Bold: CDR sequence and not bold: framework sequence) |
| KB-AT-003 | |
| domains | Sequences |
| CDR1 | SEQ ID NO: 25 |
| | ALTFSSRAW |
| CDR2 | SEQ ID NO: 26 |
| | ITGGGTTN |
| CDR3 | SEQ ID NO: 27 |
| | NGYRYTYA |
| SEQUENCE | SEQ ID NO: 28 |
| KB-AT-003 | QVQLVQSGGGLVQPGGSLRLSCAASALTFSSRAWAWYRQAPGKQREL |
| | VASITGGGTTNYADSVKGRFTISRDNAKNTVYLQMNSLKPEDTAVHY |
| | CNGYRYTYAWGQGTQVTVSS |
| | (Bold: CDR sequence and not bold: framework sequence) |
| KB-AT-004 | |
| domains | Sequences |
| CDR1 | SEQ ID NO: 29 |
| | AMTFSIR |
| CDR2 | SEQ ID NO: 30 |
| | IGTGDIT |
| CDR3 | SEQ ID NO: 31 |
| | NGYRSTYA |
| SEQUENCE | SEQ ID NO: 32 |
| KB-AT-004 | VQLQQSGGGLVQPGGSLRLSCAASAMTFSIRAWAWYRQAPGKQRELV |
| | ASIGTGDITNYADSVKGRFTISRDNAKNTFYLQMNSLKPEDTAVYYCN |
| | GYRSTYAWGQGTQVTVSS |
| | (Bold: CDR sequence and not bold: framework sequence) |
| KB-AT-005 | |
| domains | Sequences |
| CDR1 | SEQ ID NO: 33 |
| | GRDFNDAAL |
| CDR2 | SEQ ID NO: 34 |
| | ITSGGVR |
| CDR3 | SEQ ID NO: 35 |
| | KADSFKGDYDTSWYLY |
| SEQUENCE | SEQ ID NO: 36 |
| KB-AT-005 | EVQLVESGGGLVQPGGSLRLSCEASGRDFNDAALGWSRQVPGKARET |
| | VAMITSGGVRNYAETVKDRFTISRDNAKNTVYLDMNNLQPDDTGVYY |
| | CKADSFKGDYDTSWYLYWGQGTQVTVSS |
| | (Bold: CDR sequence and not bold: framework sequence) |
| KB-AT-006 | |
| domains | Sequences |
| CDR1 | SEQ ID NO: 37 |
| | GRTFSNNG |
| CDR2 | SEQ ID NO: 38 |
| | ISWSSGST |
| CDR3 | SEQ ID NO: 39 |
| | AARTRYNSGYFTRNYDY |
| SEQUENCE | SEQ ID NO: 40 |
| KB-AT-006 | QVQLQQSGGGLVQAGGSLRLSCAASGRTFSNNGMGWFRQAPGKEREF |
| | VAAISWSSGSTYYADSVKGRYTISRDNAKNTVYLQMNSLKPEDTAVY |
| | YCAARTRYNSGYFTRNYDYWGQGTQVTVSS |
| | (Bold: CDR sequence and not bold: framework sequence) |
| KB-AT-007 | |
| domains | Sequences |
| CDR1 | SEQ ID NO: 41 |
| | GRTFRNYV |
| CDR2 | SEQ ID NO: 42 |
| | INRSGAIT |
| CDR3 | SEQ ID NO: 43 |
| | AAGETTWSIRRDDYDY |
| SEQUENCE | SEQ ID NO: 44 |
| KB-AT-007 | QVQLQQSGGGLVQAGGSLRLSCAASGRTFRNYVMGWFRQAPGKDPE |
| | FIAGINRSGAITYYGDSVKGRFTISRDNAKNTVSLQMNSLEPEDTAVYY |
| | CAAGETTWSIRRDDYDYWGQGTQVTVSS |
| | (Bold: CDR sequence and not bold: framework sequence) |

In a further embodiment, the drug conjugate according to the invention wherein the isolated single domain antibody directed against AT is a biparatopic antibody.

As used herein, the term “biparatopic” antibody means a polypeptide comprising two single domain antibodies, wherein these two single domain antibodies are capable of binding to two different epitopes of one antigen (e.g. antithrombin), which epitopes are not normally bound at the same time by one monospecific immunoglobulin, such as e.g. a conventional antibody or one single domain antibody. Biparatopic polypeptide is also called as bivalent antibody. In the context of the invention, the peptide as described herein is linked to a biparatopic polypeptide against AT. In a particular embodiment, the biparatopic antibodies against antithrombin are selected from the group consisting of: KB-AT-002/003, KB-AT-001/002, KB-AT-001/003 and KB-AT-001/005.

The drug conjugate according to the invention, wherein the isolated single domain antibody comprises:
i) the sequences KB-AT-002 and one KB-AT-003 having at least 70% sequence identity with sequence set forth as SEQ ID NO: 45;
ii) the sequences KB-AT-001 and KB-AT-002 having at least 70% sequence identity with sequence set forth as SEQ ID NO: 46;
iii) the sequences KB-AT-001 and KB-AT-003 having at least 70% sequence identity with sequence set forth as SEQ ID NO: 47; or
iv) the sequences KB-AT-001 and KB-AT-005 having at least 70% sequence identity with sequence set forth as SEQ ID NO: 48.

In a particular embodiment, the drug conjugate according to the invention, wherein said isolated single domain antibody having sequence as set forth as SEQ ID NO:45 is fused to the peptide according to the invention comprising a sequence set forth as SEQ ID NO: 49.

The sequences of biparatopic polypeptide against AT and the fusion of biparatopic polypeptide KB-AT-002 and KB-AT-003 with the peptide of the invention are indicated in the following table (C):

TABLE C

| KB-AT-002/003 | Sequence |
|---|---|
| SEQUENCE | SEQ ID NO: 45 |
| KB-AT-002/003 | QVQLQESGGGLVQAGGSLRLSCAASGRTFNNNGMGWFRQAPGKERE |
| | FVAAISWSGGSTYYADSVKGRYIMSRDNAKNTVYLQMNSLKPEDTA |
| | VYYCAARTRYNSGLFSRNYDYWGQGTQVTVSSGGGSGGGSGGGSGG |
| | GSQVQLQESGGGLVQPGGSLRLSCAASALTFSSRAWAWYRQAPGKQ |
| | RELVASITGGGTTNYADSVKGRFTISRDNAKNTVYLQMNSLKPEDTA |
| | VHYCNGYRYTYAWGQGTQVTVSS |
| | Italic: (GGGS)4 linker sequence |
| SEQUENCE | SEQ ID NO: 46 |
| KB-AT-001/002 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLVQSGGGLVQAGGSLRLSCAASGRTFNNNGMGWFRQAPGKEREF |
| | VAAISWSGGSTYYADSVKGRYIMSRDNAKNTVYLQMNSLKPEDTAV |
| | YYCAARTRYNSGLFSRNYDYWGQGTQVTVSS |
| | Italic: (GGGS)4 linker sequence |
| SEQUENCE | SEQ ID NO: 47 |
| KB-AT-001/003 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLQESGGGLVQPGGSLRLSCAASALTFSSRAWAWYRQAPGKQREL |
| | VASITGGGTTNYADSVKGRFTISRDNAKNTVYLQMNSLKPEDTAVHY |
| | CNGYRYTYAWGQGTQVTQVTVSS |
| | Italic: (GGGS)4 linker sequence |
| SEQUENCE | SEQ ID NO: 48 |
| KB-AT-001/005 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSE |
| | VQLVESGGGLVQPGGSLRLSCEASGRDFNDAALGWSRQVPGKARET |
| | VAMITSGGVRNYAETVKDRFTISRDNAKNTVYLDMNNLQPDDTGVY |
| | YCKADSFKGDYDTSWYLYWGQGTQVTVSS |
| | Italic: (GGGS)4 linker sequence |
| SEQUENCE KB | SEQ ID NO: 49 |
| AT-002/003_ABP | QVQLQESGGGLVQAGGSLRLSCAASGRTFNNNGMGWFRQAPGKERE |
| | FVAAISWSGGSTYYADSVKGRYIMSRDNAKNTVYLQMNSLKPEDTA |
| | VYYCAARTRYNSGLFSRNYDYWGQGTQVTVSSGGGSGGGSGGGSGG |
| | GSQVQLQESGGGLVQPGGSLRLSCAASALTFSSRAWAWYRQAPGKQ |
| | RELVASITGGGTTNYADSVKGRFTISRDNAKNTVYLQMNSLKPEDTA |
| | VHYCNGYRYTYAWGQGTQVTVSSGRGGGSGGGSGGGSGGGSQGLIG |
| | DIALPRWGALWGDSV |

In a further embodiment, the isolated single domain antibody directed against AT is trivalent antibody. “Trivalent antibody” means a polypeptide comprising three single domain antibodies, wherein these three single domain antibodies are capable of binding to three different epitopes of one antigen (e.g. antithrombin), which epitopes are not normally bound at the same time by one monospecific immunoglobulin, such as e.g. a conventional antibody or one single domain antibody. In a particular embodiment, the trivalent antibodies against antithrombin are selected from the group consisting of: KB-AT-112, KB-AT-113 and KB-AT-115. In a particular embodiment, the fusion protein a trivalent antibody which comprises two isolated single domain antibodies KB-AT-001 according to the invention, which are linked to the isolated single domain antibody KB-AT-002 according to the invention. In a particular embodiment, the invention relates to a trivalent antibody which comprises two isolated single domain antibodies KB-AT-001 according to the invention, which are linked to the isolated single domain antibody KB-AT-003 according to the invention. In a particular embodiment, the invention relates to a trivalent antibody which comprises two isolated single domain antibodies KB-AT-001 according to the invention, which are linked to the isolated single domain antibody KB-AT-005 according to the invention.

The drug conjugate according to the invention, wherein the isolated single domain antibody comprises:
i) the two sequences of KB-AT-001 and one sequence of KB-AT-002, having at least 70% sequence identity with sequence set forth as SEQ ID NO: 50 (KB-AT-112);
ii) the two sequences of KB-AT-001 and KB-AT-003 having at least 70% sequence identity with sequence set forth as SEQ ID NO: 51 (KB-AT-113); or
iii) the two sequences of KB-AT-001 and KB-AT-005 having at least 70% sequence identity with sequence set forth as SEQ ID NO: 52 (KB-AT-115).

In a particular embodiment, the trivalent antibodies as described above are fused with the peptide of the invention.

The sequences of trivalent single domain antibody against AT are indicated in the following table (D):

TABLE D

| KB-AT-112 | Sequence |
|---|---|
| SEQUENCE | SEQ ID NO: 50 |
| KB-AT-112 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYC |
| | AAGETTWSIRRDDYDYWGQGTQVTVSSGGGSGGGSGGGSGGGSQVQ |
| | LVQSGGGLVQAGGSLRLSCAASGRTFNNNGMGWFRQAPGKEREFVA |
| | AISWSGGSTYYADSVKGRYIMSRDNAKNTVYLQMNSLKPEDTAVYY |
| | CAARTRYNSGLFSRNYDYWGQGTQVTVSS |
| | Italic: (GGGS)4 linker sequence |
| SEQUENCE | SEQ ID NO: 51 |
| KB-AT-113 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYC |
| | AAGETTWSIRRDDYDYWGQGTQVTVSSGGGSGGGSGGGSGGGSQVQ |
| | LQESGGGLVQPGGSLRLSCAASALTFSSRAWAWYRQAPGKQRELVAS |
| | ITGGGTTNYADSVKGRFTISRDNAKNTVYLQMNSLKPEDTAVHYCNG |
| | YRYTYAWGQGTQVTQVTVSS |
| | Italic: (GGGS)4 linker sequence |
| SEQUENCE | SEQ ID NO: 52 |
| KB-AT-115 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYC |
| | AAGETTWSIRRDDYDYWGQGTQVTVSSGGGSGGGSGGGSGGGSEVQ |
| | LVESGGGLVQPGGSLRLSCEASGRDFNDAALGWSRQVPGKARETVA |
| | MITSGGVRNYAETVKDRFTISRDNAKNTVYLDMNNLQPDDTGVYYC |
| | KADSFKGDYDTSWYLYWGQGTQVTVSS |
| | Italic: (GGS)4 linker sequence |

In a further embodiment, the isolated single domain antibody directed against AT is quadrivalent antibody. “Quadrivalent antibody” means a polypeptide comprising four single domain antibodies, wherein these four single domain antibodies are capable of binding to four different epitopes of one antigen (e.g. antithrombin), which epitopes are not normally bound at the same time by one monospecific immunoglobulin, such as e.g. a conventional antibody or one single domain antibody. In a particular embodiment, the quadrivalent antibody against antithrombin is KB-AT-1123. In some embodiments, the drug conjugate according to the invention comprising two sequences of KB-AT-001, one of KB-AT-002 sequence and one sequence of KB-AT-003, having at least 70% sequence identity with sequence set forth as SEQ ID NO: 53.

In a particular embodiment, the trivalent antibodies as described above are fused with the peptide of the invention.

The sequences of trivalent single domain antibody against AT are indicated in the following table (E):

TABLE E

| KB-AT-1123 | Sequence |
|---|---|
| SEQUENCE | SEQ ID NO: 53 |
| KB-AT-1123 | QVQLQESGGGLVQAGGSLRLSCAASGRTFRNYVMGWFRQAPGKDPE |
| | FIAGINRSGAITYYGDSVKGRFTISRDNAKNTVSLQMNSLEPEDTAVY |
| | YCAAGETTWSIRRDDYDYWGQGTQVTVSSGGGSGGGSGGGSGGGSQ |
| | VQLQESGGGLVQAGGSLRLSCAASGRTFRNYVMGWFRQAPGKDPEFI |
| | AGINRSGAITYYGDSVKGRFTISRDNAKNTVSLQMNSLEPEDTAVYYC |
| | AAGETTWSIRRDDYDYWGQGTQVTVSSGGGSGGGSGGGSGGGSQVQ |
| | LVQSGGGLVQAGGSLRLSCAASGRTFNNNGMGWFRQAPGKEREFVA |
| | AISWSGGSTYYADSVKGRYIMSRDNAKNTVYLQMNSLKPEDTAVYY |
| | CAARTRYNSGLFSRNYDYWGQGTQVTVSSGGGSGGGSGGGSGGGSQ |
| | VQLQESGGGLVQPGGSLRLSCAASALTFSSRAWAWYRQAPGKQREL |
| | VASITGGGTTNYADSVKGRFTISRDNAKNTVYLQMNSLKPEDTAVHY |
| | CNGYRYTYAWGQGTQVTQVTVSS |
| | Italic: (GGGS)4 linker sequence |

In a particular embodiment, the drug conjugate according to the invention, wherein the isolated single domain antibody is directed against the fibrinogen.

As used herein, the term “fibrinogen” also known as clotting factor I, refers to a glycoprotein in vertebrates. It is a glycoprotein synthesized in the liver with an apparent molecular weight of 340.000 Da, is composed of two dimers, each of them built of three pairs of non-identical polypeptide chains called Aα, Bβ and γ linked by disulfide bridges. Upon injury of blood vessels, blood platelets are activated and a plug is formed. Fibrinogen is involved in primary haemostasis by aiding cross-linking of activated platelets. In parallel activation of the clotting cascade is initiated. As the endpoint, fibrinogen is converted into fibrin by proteolytic release of fibrinopeptide A and at a slower rate fibrinopeptide B by thrombin. The soluble fibrin monomers are assembled to double stranded twisted fibrils. Subsequently these fibrils are arranged in a lateral manner, resulting in thicker fibers. These fibers are then cross-linked by FXIIIa to a fibrin network, which stabilizes the platelet plug by interactions of the fibrin with activated platelets, resulting in a stable clot. In a particular embodiment, the isolated single domain antibodies directed against fibrinogen are selected from the group consisting of: KB-FIBR-008, KB-FIBR-009, KB-FIBR-011, KB-FIBR-022 or KB-FIBR-048.

The drug conjugate according to the invention, wherein the isolated single domain antibody comprises:

i) a CDR1 having least 70% of identity with sequence set forth as SEQ ID NO:54, a CDR2 having at least 70% of identity with sequence set forth as SEQ ID NO:55 and a CDR3 having at least 70% of identity with sequence set forth as SEQ ID NO:56;

ii) a CDR1 having least 70% of identity with sequence set forth as SEQ ID NO:59, a CDR2 having at least 70% of identity with sequence set forth as SEQ ID NO:60 and a CDR3 having at least 70% of identity with sequence set forth as SEQ ID NO:61;

iii) a CDR1 having least 70% of identity with sequence set forth as SEQ ID NO:64, a CDR2 having at least 70% of identity with sequence set forth as SEQ ID NO:65 and a CDR3 having at least 70% of identity with sequence set forth as SEQ ID NO:66;

iv) a CDR1 having least 70% of identity with sequence set forth as SEQ ID NO:69, a CDR2 having at least 70% of identity with sequence set forth as SEQ ID NO:70 and a CDR3 having at least 70% of identity with sequence set forth as SEQ ID NO:71; or

v) a CDR1 having least 70% of identity with sequence set forth as SEQ ID NO:74, a CDR2 having at least 70% of identity with sequence set forth as SEQ ID NO:75 and a CDR3 having at least 70% of identity with sequence set forth as SEQ ID NO:76.

In a particular embodiment, the drug conjugate according to the invention, wherein said isolated single domain antibody is fused to the peptide according to the invention comprising a sequence set forth as SEQ ID NO: 58, SEQ ID NO: 63, SEQ ID NO: 68, SEQ ID NO: 73 or SEQ ID NO: 78.

The sequences of KB-FIBR-008, KB-FIBR-009, KB-FIBR-011, KB-FIBR-022 or KB-FIBR-048 and their fusion with the peptide of the invention are indicated in the following table (F):

TABLE F

| KB-FIBR-008 | |
|---|---|
| domains | Sequences |
| CDR1 | SEQ ID NO: 54 |
| | GGSMTSGYAA |
| CDR2 | SEQ ID NO: 55 |
| | AYDGSTYYSPSLES |
| CDR3 | SEQ ID NO: 56 |
| | RLYNGFEH |
| SEQUENCE KB- | SEQ ID NO: 57 |
| FIBR-008 | QVQLQESGPGLVKPSQTLSLTCTVSGGSMTSGYAAWNWIRQPPGKGLEWM |
| | GIIAYDGSTYYSPSLESRTSISRDTSKNQFSLQLSSVTPEDTAVYYCARLYNGF |
| | EHWGQGTQVTVSS |
| | (Bold: CDR sequence and not bold: framework sequence) |
| SEQUENCE | Sequences |
| KB-FIBR-008bv- | SEQ ID NO: 58 |
| ABP | QVQLVQSGGGLVQAGDSLRLSCAASGGTFSNYAMGWFRQTPGKEREFVARI |
| | SRSGHRTDYADSAKGRFTISRDNAKNTVYLQMNSLKPEDTAVYYCAARSDW |
| | SIATTATSYDYWGQGTQVTVSSGGGSGGGSGGGSGGGSQVQLVQSGGGLV |
| | QAGDSLRLSCAASGGTFSNYAMGWFRQTPGKEREFVARISRSGHRTDYADS |
| | AKGRFTISRDNAKNTVYLQMNSLKPEDTAVYYCAARSDWSIATTATSYDYWG |
| | QGTQVTVSSGRGGGSLTPRGVRLGGGSQGLIGDIALPRWGALWGDSV |
| | Italic: linker sequence; Underlined: thrombin-cleavage |
| | site; Bold: ABP |
| KB-FIBR-009 | |
| domains | Sequences |
| CDR1 | SEQ ID NO: 59 |
| | GFTFDDYAMS |
| CDR2 | SEQ ID NO: 60 |
| | ISWNGKTTLYAESMKG |
| CDR3 | SEQ ID NO: 61 |
| | RGSGLFVS |
| SEQUENCE KB- | SEQ ID NO: 62 |
| FIBR-009 | QLVESGGGLVQPGGSLRLSCAASGFTFDDYAMSWVRQAPGKGL |
| | EWVSAISWNGKTTLYAESMKGRFTISRDNAKNTLYLQMNNLKP |
| | EDTAVYSCADRGSGLFVSWGQGTQVTVSS |
| | (Bold: CDR sequence and not bold: framework sequence) |
| SEQUENCE | Sequences |
| KB-FIBR-009bv- | SEQ ID NO: 63 |
| ABP | QLVESGGGLVQPGGSLRLSCAASGFTFDDYAMSWVRQAPGKGL |
| | EWVSAISWNGKTTLYAESMKGRFTISRDNAKNTLYLQMNNLKPE |
| | DTAVYSCADRGSGLFVSWGQGTQVTVSSGGGSGGGSGGGSGGGS |
| | QLVESGGGLVQPGGSLRLSCAASGFTFDDYAMSWVRQAPGKGL |
| | EWVSAISWNGKTTLYAESMKGRFTISRDNAKNTLYLQMNNLKPE |
| | DTAVYSCADRGSGLFVSWGQGTQVTVSSGRGGGSLTPRGVRLGGG |
| | SQGLIGDIALPRWGALWGDSV |
| | Italic: linker sequence; Underlined: thrombin-cleavage |
| | site; Bold: ABP |
| KB-FIBR-011 | |
| domains | Sequences |
| CDR1 | SEQ ID NO: 64 |
| | GFTFGSYDMN |
| CDR2 | SEQ ID NO: 65 |
| | ISYSTWYADSMEG |
| CDR3 | SEQ ID NO: 66 |
| | DLVGLVGLEGGY |
| SEQUENCE KB- | SEQ ID NO: 67 |
| FIBR-011 | EVQLVESGGGLVQPGGSLRLSCAASGFTFGSYDMNWVRQAPGKGPEWVSTI |
| | SYSTWYADSMEGRFTISRDNAKNTLYLQMTSLKPEDTAVYYCTTDLVGLVGL |
| | EGGYWGQGTQVTVSS |
| | (Bold: CDR sequence and not bold: framework sequence) |
| SEQUENCE | SEQ ID NO: 68 |
| KB-FIBR- | EVQLVESGGGLVQPGGSLRLSCAASGFTFGSYDMNWVRQAPGKGPEWVSTI |
| 011bv_ABP | SYSTWYADSMEGRFTISRDNAKNTLYLQMTSLKPEDTAVYYCTTDLVGLVGL |
| | EGGYWGQGTQVTVSSGGGSGGGSGGGSGGGSEVQLVESGGGLVQPGGSL |
| | RLSCAASGFTFGSYDMNVVVRQAPGKGPEWVSTISYSTWYADSMEGRFTISR |
| | DNAKNTLYLQMTSLKPEDTAVYYCTTDLVGLVGLEGGYWGQGTQVTVSSGR |
| | GGGSLTPRGVRLGGGSQGLIGDIALPRWGALWGDSV |
| | Italic: linker sequence; Underlined: thrombin-cleavage |
| | site; Bold: ABP |
| KB-FIBR-022 | |
| domains | Sequences |
| CDR1 | SEQ ID NO: 69 |
| | GFTFDDYAMS |
| CDR2 | SEQ ID NO: 70 |
| | INSGGGSTSYADSVKG |
| CDR3 | SEQ ID NO: 71 |
| | KIWTQFGY |
| SEQUENCE KB- | SEQ ID NO: 72 |
| FIBR-022 | QVQLVQSGGGLVQPGGSLRLSCAASGFTFDDYAMSWVRQAPGKGLEWVSA |
| | INSGGGSTSYADSVKGRFTISRDNDKKTVYLQMNSLKPEDTAVYYCAVKIWT |
| | QFGYWGQGTQVTVSS |
| | (Bold: CDR sequence and not bold: framework sequence) |
| SEQUENCE | SEQ ID NO: 73 |
| KB-FIBR- | QVQLVQSGGGLVQPGGSLRLSCAASGFTFDDYAMSWVRQAPGKGLEWVSA |
| 022bv_ABP | INSGGGSTSYADSVKGRFTISRDNDKKTVYLQMNSLKPEDTAVYYCAVKIWTQ |
| | FGYVVGQGTQVTVSSGGGSGGGSGGGSGGGSQVQLVQSGGGLVQPGGSL |
| | RLSCAASGFTFDDYAMSWVRQAPGKGLEWVSAINSGGGSTSYADSVKGRFTI |
| | SRDNDKKTVYLQMNSLKPEDTAVYYCAVKIWTQFGYWGQGTQVTVSSGRGG |
| | GSLTPRGVRLGGGSQGLIGDIALPRWGALWGDSV |
| | Italic: linker sequence; Underlined: thrombin-cleavage |
| | site; Bold: ABP |
| KB-FIBR-048 | |
| domains | Sequences |
| CDR1 | SEQ ID NO: 74 |
| | KGIFAFNGMG |
| CDR2 | SEQ ID NO: 75 |
| | IMQPNGRTIYKDTVKG |
| CDR3 | SEQ ID NO: 76 |
| | WVNRTNDMY |
| SEQUENCE KB- | SEQ ID NO: 77 |
| FIBR-048 | QVQLVQSGGGSVQPGGSLRLSCAASKGIFAFNGMGWFRQAPGKERELVAK |
| | MQPNGRTIYKDTVKGRFTISRDIQKATVDLLMKSLQPEDTADYYCGAWVNRT |
| | NDMYWGQGTQVTVSS |
| | (Bold: CDR sequence and not bold: framework sequence) |
| SEQUENCE | SEQ ID NO: 78 |
| KB-FIBR- | QVQLVQSGGGSVQPGGSLRLSCAASKGIFAFNGMGWFRQAPGKERELVAK |
| 048bv_ABP | MQPNGRTIYKDTVKGRFTISRDIQKATVDLLMKSLQPEDTADYYCGAWVNRT |
| | NDMYWGQGTQVTVSSGGGSGGGSGGGSGGGSQVQLVQSGGGSVQPGGS |
| | LRLSCAASKGIFAFNGMGWFRQAPGKERELVAKMQPNGRTIYKDTVKGRFTI |
| | SRDIQKATVDLLMKSLQPEDTADYYCGAVVVNRTNDMYWGQGTQVTVSSGR |
| | GGGSLTPRGVRLGGGSQGLIGDIALPRWGALWGDSV |
| | Italic: linker sequence; Underlined: thrombin-cleavage |
| | site; Bold: ABP |

In a particular embodiment, the drug conjugate according to the invention, wherein the isolated single domain antibody is directed against the plasminogen activator inhibitor-1 (PAI-1).

As used herein, the term “Plasminogen Activator inhibitor type-1 (PAI-1)” also known as endothelial plasminogen activator inhibitor or serpin E1 refers to a protein that in humans is encoded by the SERPINE1 gene. It is the main inhibitor of tissue-type plasminogen activator (tPA) and urokinase-type plasminogen activator (uPA), the key serine proteases responsible for plasmin generation. PAI-1 regulates fibrinolysis by inhibiting plasminogen activation in the vascular compartment. ene is localized to chromosome 7, consists of eight introns and nine exons, and has a size of 12, 169 b (Klinger, W. ei as. Proc. Nail Acad. Sci. USA 84: 8548, 1987). PAI-1 is a single chai glycoprotein of approximately 50 kDa (379 amino acids) from the SERPEN (serine protease inhibitor) superfamily that is synthesized in the active conformation but spontaneously becomes latent in the absence of vitronectin (Vn). Vitronectin, the mam cefaclor of PAI-1, stabilizes the active conformation with the Reactive Center Loop (RCL) which is approximately 20 amino acids that are exposed on the surface. In a particular embodiment, the isolated single domain antibodies directed against PAI-1 are selected from the group consisting of: KB-PAI1-002; KB-PAI1-006 and KB-PAI1-007.

The drug conjugate according to the invention, wherein the isolated single domain antibody comprises:

i) a CDR1 having least 70% of identity with sequence set forth as SEQ ID NO:79, a CDR2 having at least 70% of identity with sequence set forth as SEQ ID NO:80 and a CDR3 having at least 70% of identity with sequence set forth as SEQ ID NO:81; wherein said isolated single domain antibody has the sequence set forth as SEQ ID NO:82;

ii) a CDR1 having least 70% of identity with sequence set forth as SEQ ID NO:84, a CDR2 having at least 70% of identity with sequence set forth as SEQ ID NO:85 and a CDR3 having at least 70% of identity with sequence set forth as SEQ ID NO:86; or

iii) a CDR1 having least 70% of identity with sequence set forth as SEQ ID NO:89, a CDR2 having at least 70% of identity with sequence set forth as SEQ ID NO:90 and a CDR3 having at least 70% of identity with sequence set forth as SEQ ID NO:91.

In a particular embodiment, the drug conjugate according to the invention, wherein said isolated single domain antibody is fused to the peptide according to the invention comprising a sequence set forth as SEQ ID NO: 83, SEQ ID NO: 88 or SEQ ID NO: 93.

The sequences of KB-PAI1-002, KB-PAI1-006, KB-PAI1-007 and their fusion with the peptide of the invention are indicated in the following table (G):

TABLE G

| KB-PAI1-002 | |
|---|---|
| domains | Sequences |
| CDR1 | SEQ ID NO: 79 |
| | MSSFGMYHMG |
| CDR2 | SEQ ID NO: 80 |
| | IGPSGNTYYLDSVKG |
| CDR3 | SEQ ID NO: 81 |
| | SRKVVSLAVDY |
| SEQUENCE KB- | SEQ ID NO: 82 |
| PAI1-002 | EVQLVESGGGLVQTGGSLRLSCVASMSSFGMYHMGWYRQAPGSQRKMVA |
| | WIGPSGNTYYLDSVKGRFTISRDNAKNTLYLQMNSLKPEDTAVYYCAKSRKV |
| | VSLAVDYRGQGTQVTVSS |
| | (Bold: CDR sequence and not bold: framework sequence) |
| SEQUENCE | SEQ ID NO: 83 |
| KB-PAI1- | EVQLVESGGGLVQTGGSLRLSCVASMSSFGMYHMGWYRQAPGSQRKMVA |
| 002bv_ABP | WIGPSGNTYYLDSVKGRFTISRDNAKNTLYLQMNSLKPEDTAVYYCAKSRKVV |
| | SLAVDYRGQGTQVTVSSGGGSGGGSGGGSGGGSEVQLVESGGGLVQTGG |
| | SLRLSCVASMSSFGMYHMGWYRQAPGSQRKMVAWIGPSGNTYYLDSVKGR |
| | FTISRDNAKNTLYLQMNSLKPEDTAVYYCAKSRKVVSLAVDYRGQGTQVTVS |
| | SGRGGGSLTPRGVRLGGGSQGLIGDIALPRWGALWGDSV |
| | Italic: linker sequence; Underlined: thrombin-cleavage |
| | site; Bold: ABP |
| KB-PAI1-006 | |
| domains | Sequences |
| CDR1 | SEQ ID NO: 84 |
| | GDRLSAYAMG |
| CDR2 | SEQ ID NO: 85 |
| | ISRTSGRTYYAGSVKG |
| CDR3 | SEQ ID NO: 86 |
| | RYGRYDVARMSRVDY |
| SEQUENCE KB- | SEQ ID NO: 87 |
| PAI1-006 | QVQLVQSGGGLVQAGESLRLSCAASGDRLSAYAMGWFRQGPGKDREFVSA |
| | ISRTSGRTYYAGSVKGRFTISRDNAKNTVYLQMNSLKAEDTAVYYCAARYGR |
| | YDVARMSRVDYWGQGTQVTVSS |
| | (Bold: CDR sequence and not bold: framework sequence) |
| SEQUENCE | SEQ ID NO: 88 |
| KB-PAI1- | QVQLVQSGGGLVQAGESLRLSCAASGDRLSAYAMGWFRQGPGKDREFVSAI |
| 006bv_ABP | SRTSGRTYYAGSVKGRFTISRDNAKNTVYLQMNSLKAEDTAVYYCAARYGRY |
| | DVARMSRVDYWGQGTQVTVSSGGGSGGGSGGGSGGGSQVQLVQSGGGL |
| | VQAGESLRLSCAASGDRLSAYAMGWFRQGPGKDREFVSAISRTSGRTYYAG |
| | SVKGRFTISRDNAKNTVYLQMNSLKAEDTAVYYCAARYGRYDVARMSRVDY |
| | WGQGTQVTVSSGRGGGSLTPRGVRLGGGSQGLIGDIALPRWGALWGDSV |
| | Italic: linker sequence; Underlined: thrombin-cleavage |
| | site; Bold: ABP |
| KB-PAI1-007 | |
| domains | Sequences |
| CDR1 | SEQ ID NO: 89 |
| | GFTFSSHWMN |
| CDR2 | SEQ ID NO: 90 |
| | ISGGGFTTYYADFVKG |
| CDR3 | SEQ ID NO: 91 |
| | GPWQDVDA |
| SEQUENCE KB- | SEQ ID NO: 92 |
| PAI1-007 | EVQLVQSGGGLVQPGGSLRLSCAASGFTFSSHWMNWVRQAPGKGLQWISTI |
| | SGGGFTTYYADFVKGRFTISRDNAKNTLYLQMNSLKSEDTAVYYCAKGPWQD |
| | VDAWGQGTQVTVSS |
| | (Bold: CDR sequence and not bold: framework sequence) |
| SEQUENCE | SEQ ID NO: 93 |
| KB-PAI1- | EVQLVQSGGGLVQPGGSLRLSCAASGFTFSSHWMNWVRQAPGKGLQWISTI |
| 007bv_ABP | SGGGFTTYYADFVKGRFTISRDNAKNTLYLQMNSLKSEDTAVYYCAKGPWQD |
| | VDAWGQGTQVTVSSGGGSGGGSGGGSGGGSEVQLVQSGGGLVQPGGSLR |
| | LSCAASGFTFSSHWMNVVVRQAPGKGLQWISTISGGGFTTYYADFVKGRFTIS |
| | RDNAKNTLYLQMNSLKSEDTAVYYCAKGPWQDVDAWGQGTQVTVSSGRGG |
| | GSLTPRGVRLGGGSQGLIGDIALPRWGALWGDSV |
| | Italic: linker sequence; Underlined: thrombin-cleavage |
| | site; Bold: ABP |

In a particular embodiment, the drug conjugate according to the invention comprising a thrombin-cleavage site. Typically, the thrombin-cleavage site comprising an amino acid sequence LTPRGVRL (SEQ ID NO: 94).

The drug conjugate according to the invention, wherein the isolated single domain antibody comprises:
i) a CDR1 having at least 70% of identity with sequence set forth as SEQ ID NO:2, a CDR2 having at least 70% of identity with sequence set forth as SEQ ID NO:3 and a CDR3 having at least 70% of identity with sequence set forth as SEQ ID NO:4;
ii) a CDR1 having at least 70% of identity with sequence set forth as SEQ ID NO:7, a CDR2 having at least 70% of identity with sequence set forth as SEQ ID NO:8 and a CDR3 having at least 70% of identity with sequence set forth as SEQ ID NO:9;
iii) a CDR1 having at least 70% of identity with sequence set forth as SEQ ID NO:12, a CDR2 having at least 70% of identity with sequence set forth as SEQ ID NO:13 and a CDR3 having at least 70% of identity with sequence set forth as SEQ ID NO:14;
iv) a CDR1 having at least 70% of identity with sequence set forth as SEQ ID NO:17, a CDR2 having at least 70% of identity with sequence set forth as SEQ ID NO:18 and a CDR3 having at least 70% of identity with sequence set forth as SEQ ID NO:19;
v) a CDR1 having at least 70% of identity with sequence set forth as SEQ ID NO:21, a CDR2 having at least 70% of identity with sequence set forth as SEQ ID NO:22 and a CDR3 having at least 70% of identity with sequence set forth as SEQ ID NO:23;
vi) a CDR1 having at least 70% of identity with sequence set forth as SEQ ID NO:25, a CDR2 having at least 70% of identity with sequence set forth as SEQ ID NO:26 and a CDR3 having at least 70% of identity with sequence set forth as SEQ ID NO:27;
vii) a CDR1 having at least 70% of identity with sequence set forth as SEQ ID NO:29, a CDR2 having at least 70% of identity with sequence set forth as SEQ ID NO:30 and a CDR3 having at least 70% of identity with sequence set forth as SEQ ID NO:31;
viii) a CDR1 having at least 70% of identity with sequence set forth as SEQ ID NO:33, a CDR2 having at least 70% of identity with sequence set forth as SEQ ID NO:34 and a CDR3 having at least 70% of identity with sequence set forth as SEQ ID NO:35;
ix) a CDR1 having at least 70% of identity with sequence set forth as SEQ ID NO:37, a CDR2 having at least 70% of identity with sequence set forth as SEQ ID NO:38 and a CDR3 having at least 70% of identity with sequence set forth as SEQ ID NO:39;
x) a CDR1 having at least 70% of identity with sequence set forth as SEQ ID NO:41, a CDR2 having at least 70% of identity with sequence set forth as SEQ ID NO:42 and a CDR3 having at least 70% of identity with sequence set forth as SEQ ID NO:43;
xi) a CDR1 having at least 70% of identity with sequence set forth as SEQ ID NO:54, a CDR2 having at least 70% of identity with sequence set forth as SEQ ID NO:55 and a CDR3 having at least 70% of identity with sequence set forth as SEQ ID NO:56;
xii) a CDR1 having at least 70% of identity with sequence set forth as SEQ ID NO:59, a CDR2 having at least 70% of identity with sequence set forth as SEQ ID NO:60 and a CDR3 having at least 70% of identity with sequence set forth as SEQ ID NO:61;
xiii) a CDR1 having at least 70% of identity with sequence set forth as SEQ ID NO:64, a CDR2 having at least 70% of identity with sequence set forth as SEQ ID NO:65 and a CDR3 having at least 70% of identity with sequence set forth as SEQ ID NO:66;
xiv) a CDR1 having at least 70% of identity with sequence set forth as SEQ ID NO:69, a CDR2 having at least 70% of identity with sequence set forth as SEQ ID NO:70 and a CDR3 having at least 70% of identity with sequence set forth as SEQ ID NO:71;
xv) a CDR1 having at least 70% of identity with sequence set forth as SEQ ID NO:74, a CDR2 having at least 70% of identity with sequence set forth as SEQ ID NO:75 and a CDR3 having at least 70% of identity with sequence set forth as SEQ ID NO:76;
xvi) a CDR1 having at least 70% of identity with sequence set forth as SEQ ID NO:79, a CDR2 having at least 70% of identity with sequence set forth as SEQ ID NO:80 and a CDR3 having at least 70% of identity with sequence set forth as SEQ ID NO:81;
xvii) a CDR1 having at least 70% of identity with sequence set forth as SEQ ID NO:84, a CDR2 having at least 70% of identity with sequence set forth as SEQ ID NO:85 and a CDR3 having at least 70% of identity with sequence set forth as SEQ ID NO:86; or
xviii) a CDR1 having at least 70% of identity with sequence set forth as SEQ ID NO:89, a CDR2 having at least 70% of identity with sequence set forth as SEQ ID NO:90 and a CDR3 having at least 70% of identity with sequence set forth as SEQ ID NO:91.

The drug conjugate according to the invention, wherein the isolated single domain antibody comprises:
i) a CDR1 having sequence set forth as SEQ ID NO:2, a CDR2 having sequence set forth as SEQ ID NO:3 and a CDR3 having sequence set forth as SEQ ID NO:4;
ii) a CDR1 having sequence set forth as SEQ ID NO:7, a CDR2 having sequence set forth as SEQ ID NO:8 and a CDR3 having sequence set forth as SEQ ID NO:9;
iii) a CDR1 having sequence set forth as SEQ ID NO:12, a CDR2 having sequence set forth as SEQ ID NO:13 and a CDR3 having a sequence set forth as SEQ ID NO:14;
iv) a CDR1 having a sequence set forth as SEQ ID NO:17, a CDR2 having a sequence set forth as SEQ ID NO:18 and a CDR3 a sequence set forth as SEQ ID NO:19;
v) a CDR1 having sequence set forth as SEQ ID NO:21, a CDR2 having sequence set forth as SEQ ID NO:22 and a CDR3 having sequence set forth as SEQ ID NO:23;
vi) a CDR1 having sequence set forth as SEQ ID NO:25, a CDR2 having sequence set forth as SEQ ID NO:26 and a CDR3 having sequence set forth as SEQ ID NO:27;
vii) a CDR1 having sequence set forth as SEQ ID NO:29, a CDR2 having sequence set forth as SEQ ID NO:30 and a CDR3 having sequence set forth as SEQ ID NO:31;
viii) a CDR1 having sequence set forth as SEQ ID NO:33, a CDR2 having sequence set forth as SEQ ID NO:34 and a CDR3 having sequence set forth as SEQ ID NO:35;
ix) a CDR1 having sequence set forth as SEQ ID NO:37, a CDR2 having sequence set forth as SEQ ID NO:38 and a CDR3 having a sequence set forth as SEQ ID NO:39;
x) a CDR1 having sequence set forth as SEQ ID NO:41, a CDR2 having sequence set forth as SEQ ID NO:42 and a CDR3 having sequence set forth as SEQ ID NO:43;
xi) a CDR1 having sequence set forth as SEQ ID NO:54, a CDR2 having sequence set forth as SEQ ID NO:55 and a CDR3 having sequence set forth as SEQ ID NO:56;
xii) a CDR1 having sequence set forth as SEQ ID NO:59, a CDR2 having sequence set forth as SEQ ID NO:60 and a CDR3 having sequence set forth as SEQ ID NO:61;
xiii) a CDR1 having sequence set forth as SEQ ID NO:64, a CDR2 having sequence set forth as SEQ ID NO:65 and a CDR3 having sequence set forth as SEQ ID NO:66;
xiv) a CDR1 having sequence set forth as SEQ ID NO:69, a CDR2 having sequence set forth as SEQ ID NO:70 and a CDR3 having sequence set forth as SEQ ID NO:71;
xv) a CDR1 having sequence set forth as SEQ ID NO:74, a CDR2 having sequence set forth as SEQ ID NO:75 and a CDR3 having a sequence set forth as SEQ ID NO:76;
xvi) a CDR1 having sequence set forth as SEQ ID NO:79, a CDR2 having sequence set forth as SEQ ID NO:80 and a CDR3 having sequence set forth as SEQ ID NO:81;
xvii) a CDR1 having sequence set forth as SEQ ID NO:84, a CDR2 having sequence set forth as SEQ ID NO:85 and a CDR3 having sequence set forth as SEQ ID NO:86;
xviii) a CDR1 having sequence set forth as SEQ ID NO:89, a CDR2 having sequence set forth as SEQ ID NO:90 and a CDR3 having sequence set forth as SEQ ID NO:91.

The drug conjugate according to the invention, comprises:
i) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO:5;
ii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 10;
iii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 15;
iv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 20;
v) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 24;
vi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 28;
vii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 32;
viii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 36;
ix) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 40;
x) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 44;
xi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 57;
xii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 62;
xiii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 67;
xiv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 72;
xv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 77;
xvi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 82;
xvii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 87; or
xviii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ: 92.

The drug conjugate according to the invention, wherein, the isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% with the sequence set forth as SEQ ID NO:5, SEQ: 10; SEQ: 15; SEQ ID NO:20; SEQ ID NO:24; SEQ ID NO:28; SEQ: 32; SEQ: 36; SEQ: 40; SEQ: 44; SEQ: 57; SEQ: 62; SEQ: 67; as SEQ: 72; SEQ: 77; SEQ: 82; SEQ: 87; or SEQ: 92; wherein the variability of the single domain antibody is in the framework of the antibody.

In a third aspect, the invention relates to a vector which comprises the peptide or the drug conjugate of the present invention.

Typically the peptide or drug conjugate may be delivered in association with a vector. The peptide or drug conjugate of the present invention is included in a suitable vector, such as a plasmid, cosmid, episome, artificial chromosome, phage or a viral vector. So, a further object of the invention relates to a vector comprising a single domain antibodies or drug conjugate of the invention. Typically, the vector is a viral vector, which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein that allows transduction of cells of human and other species. Typically, the nucleic acid molecule or the vector of the present invention include “control sequences”’, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

### Therapeutics Methods

In a fourth aspect, the invention relates to a method of extending or increasing the half-life and the level of an endogenous protein in a subject comprising a step of adding to the said subject the drug conjugate according to the invention which is inserted or not in to a vector.

Typically, the drug conjugates of the invention are suitable for extending or increasing the half-life of an endogenous protein.

As used herein, the term “half-life” refers to the time required for a quantity to reduce to half its initial value. Half-life may be represented by the time required for half the quantity administered to a subject to be cleared from the circulation and/or other tissues in the animal. When a clearance curve of a given polypeptide is constructed as a function of time, the curve is usually biphasic with a rapid, α-phase and longer β-phase. For example, the half-life of a human VWF is 16 hours (Goudemand et al 2005). In the context of the invention, the drug conjugate as described above increases the half-life of the endogenous VWF in a subject compared to the half-life of endogenous VWF in the absence of the drug conjugate.

As used herein, the term “level” refers to an amount or a concentration of an endogenous protein in the blood stream.

As used herein, the term “endogenous protein” refers to the native protein normally found in its natural location in the subject.

Peptide without altering the overall conformation and function of the peptide or the drug conjugate as described above where such changes do not alter the biological activities of endogenous protein, lead only to extend or increase the half-life and the level of aid endogenous protein.

In a particular embodiment, the drug conjugates according to the invention are inserted or not in a vector for extending or increasing the half-life and the level of an endogenous protein.

In one embodiment, the drug conjugate of the invention is PEGylated. For example, the drug conjugate comprising an isolated single domain antibody against VWF is PEGylated (PEGrVWF).

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Other possibilities of modifications to prolong the half-life of a protein are HEPylation, polysialylation or the attachment of XTEN-polypeptides.

In a fifth aspect, the invention relates to a method of treating a subject in need thereof comprising a step of administering to said subject a therapeutically effective amount of the drug conjugate according to the invention.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. Particularly, the subject is a human. More particularly, the subject is a human afflicted with or susceptible to be afflicted with bleeding disorders.

The method according to the invention, wherein the subject suffers from deficiencies, abnormal level or structural abnormalities of at least one protein selected from the group consisting of: Von Willebrand factor, Fibrinogen, Factor II (prothrombin), Factor V, Factor VII, Factor VIII, Factor IX, Factor X, Factor XI, Factor XII, Factor XIII, Protein C, Protein S, Protein Z, Protein Z-inhibitor, Tissue factor pathway inhibitor (TFPI), α1-antitrypsin inhibitor, Thrombin Activatable Fibrinolysis Inhibitor (TAFI)/carboxypeptidase B2, Antithrombin, α2-antiplasmin, Plasmin Activator Inhibitor-1 (PAI-1), Plasminogen, tissue plasminogen activator (tPA), urinary plasminogen activator (uPA), ADAMTS13, Complement protein C2, Complement protein C3, Complement protein C4, Complement protein C6, Complement factor H, Complement factor I, Properdin, Ceruloplasmin, Kininogen, Thrombopoietin, Erytropoeitin, Soluble Mannose-binding lectin, Interferon-α, Interferon-β, Interferon-γ, Granulocyte colony-stimulating factor, Granulocyte-macrophage colony-stimulating factor, Keratinocyte growth factor, Interleukin-2, Interleukin-6, Interleukin-7, Interleukin-10, Interleukin-11, Interleukin-12, Interleukin-15, Interleukin-21, Growth hormone-releasing hormone and Hyaluronidase.

In a particular embodiment, the subject suffers from the bleeding disorders.

As used herein, the term “bleeding disorders” refers to any disorders associated with excessive bleeding, such as a congenital coagulation disorder, an acquired coagulation disorder, administration of an anticoagulant, or a trauma induced hemorrhagic condition. Bleeding disorders may include, but are not limited to, hemophilia A, hemophilia B, von Willebrand disease, idiopathic thrombocytopenia, a deficiency of one or more contact factors, such as Factor XI, Factor XII, prekallikrein, and high molecular weight kininogen (HMWK), a deficiency of one or more factors associated with clinically significant bleeding, such as Factor V, Factor VII, Factor VIII, Factor IX, Factor X, Factor XIII, Factor II (hypoprothrombinemia), and von Willebrand factor, a vitamin K deficiency, a disorder of fibrinogen, including afibrinogenemia, hypo fibrinogenemia, and dysfibrinogenemia, an alpha2-antiplasmin deficiency, and excessive bleeding such as caused by liver disease, renal disease, thrombocytopenia, platelet dysfunction, hematomas, internal hemorrhage, hemarthroses, surgery, trauma, hypothermia, menstruation, and pregnancy.

Typically, in the context of the invention, the drug conjugates as described above are suitable to increase the level of endogenous proteins involved in the coagulation and to reduce or stop the excessive bleeding.

In some embodiments, the present invention relates to a method for preventing or treating heparin induced hemorrhages in a subject in need thereof, comprising administering to said subject a therapeutically effective amount of the drug conjugate or the vector comprising the drug conjugate according to the invention.

Heparin is a widely used injectable blood thinner. It is used to treat and prevent deep vein thrombosis and pulmonary embolism. Heparin is a polymer of varying chain size. Unfractionated heparin (UFH) as a pharmaceutical is heparin that has not been fractionated to sequester the fraction of molecules with low molecular weight. In contrast, low-molecular-weight heparin (LMWH) has undergone fractionation for the purpose of making its pharmacodynamics more predictable. The term “heparin induced hemorrhages” refers to the bleeding which is a major side effect of heparin when it is administered therapeutically.

By a “therapeutically effective amount” is meant a sufficient amount of the polypeptide (or the vector containing the polypeptide) to prevent for use in a method for the treatment of bleeding disorders at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 100 mg/kg of body weight per day.

### Pharmaceutical Composition

In a sixth aspect, the invention relates to a pharmaceutical composition comprising the peptide or the drug conjugate according to the present invention, which is inserted or not in to a vector. The single-domain antibodies and drug conjugate of the invention (or the vector comprising single domain antibodies or the drug conjugate) may be combined with pharmaceutically acceptable excipients, and optionally sustained-release matrices, such as biodegradable polymers, to form pharmaceutical compositions. As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The peptide or the drug conjugate (or the vector comprising peptide or the drug conjugate) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The drug conjugate (or the vector containing the drug conjugate) may be formulated within a therapeutic mixture to comprise about 0.0001 to 1.0 milligrams, or about 0.001 to 0.1 milligrams, or about 0.1 to 1.0 or even about 100 milligrams per dose. Multiple doses can also be administered. The invention will be further illustrated by the following figures and examples.

The invention will be further illustrated by the following figures and examples. However, these examples and figures should not be interpreted in any way as limiting the scope of the present invention.

### FIGURES

FIG. 1: Binding of KB-AT-002/003_ABP to human and murine albumin in an immunosorbent assay. A construct was established encoding KB-AT-002/003 fused to an albumin-binding peptide (KB-AT-002/003_ABP). In one type of experiments, KB-AT-002/003_ABP (0-10 mg/ml) was added to wells coated with human or murine albumin (10 microgram/ml). Bound KB-AT-002/003_ABP was probed using peroxidase-labeled monoclonal anti-His tag antibodies and detected via 3,3′,5,5′-tetramethylbenzidine-hydrolysis. In FIG. 1, the observed optical density (OD) at 450 nm is plotted versus the KB-AT-002/003_AlbBP concentration. KB-AT-002/003_AlbBP is able to interact with both human and murine albumin. Data represent mean±SD of 3 determinations.

FIG. 2: Binding of KB-AT-002/003_ABP to immobilized biotinylated human albumin. Bio layer interferometry analysis was performed to assess the interaction between KB-AT-002/003_ABP and biotinylated human albumin. An example of such experiment is represented in FIG. 2. KB-AT-002/003_ABP shows dose-dependent and reversible binding to human biotinylated albumin.

FIG. 3: Binding of KB-VWF-013bv_ABP to immobilized biotinylated human or murine albumin. Bio layer interferometry analysis was performed to assess the interaction between KB-VWF-013bv_ABP and biotinylated human or murine albumin. Examples of such experiments are represented in FIG. 3A (human albumin) and FIG. 3B (murine albumin). KB-VWF-013bv_ABP shows dose-dependent and reversible binding to both human and murine biotinylated albumin.

FIG. 4: Binding of murine albumin to a complex of antithrombin and KB-AT-002/003_AlbBP. In another type of experiments, biolayer interferometry analysis was performed to determine association of albumin to a preformed antithrombin/KB-AT-002/003_ABP complex. An example of such experiment is represented in FIG. 4. Following covalent immobilization of antithrombin to amine-reactive sensors, KB-AT-002/003_ABP associates to immobilized antithrombin (Line A). Subsequently, addition of murine albumin results in an association of albumin to the preformed antithrombin/KB-AT-002/003_ABP complex (Line A). In the final phase, dissociation of the complex occurs (Line A). Line B: No KB-AT-002/003_ABP was added to antithrombin-coated biosensors. Subsequently, no binding of albumin to antithrombin-coated biosensors in the absence of KB-AT-002/003_ABP could be detected.

FIG. 5: Clearance of human von Willebrand factor in the presence or absence of KB-VWF-013bv_ABP. Purified plasma-derived human VWF (0.25 mg/kg) was given intravenously in the absence or presence of KB-VWF-013bv_ABP (0.63 mg/kg; panel A or 1.7 mg/kg; panel B) to wild-type C57B6/J mice. At indicated time-points, blood was collected to prepare plasma and residual VWF antigen was measured using an in-house ELISA specific for human VWF. Plotted are residual VWF antigen levels (percentage of the amount injected) versus time after injection (hours). VWF that is given in the presence of KB-VWF-013bv_ABP is removed from the circulation remarkably slower compared to VWF that is injected alone. Data represent mean±SD of 3-18 mice.

FIG. 6: Increasing endogenous VWF plasma levels via injection of KB-VWF-013bv_ABP. Wild-type C57B6/J mice were given a single intravenous dose of vehicle (PBS) or KB-VWF-013bv_ABP (2.5 mg/kg; panel A) via the lateral tail vein. Two mice were given the same dose of KB-VWF-013bv_ABP subcutaneously (panel B). Blood was collected 24 h before injection and at indicated time-points after injection to prepare plasma and to measure endogenous murine VWF antigen levels. VWF antigen levels measured in pre-injection samples were considered as VWF antigen levels at t=0, and were arbitrarily set at 100%. In contrast to mice treated with vehicle, mice injected intravenously with KB-VWF-013bv_ABP are characterized by a >10-fold increase in VWF antigen levels when measured three days after injection. Both mice receiving KB-VWF-013bv_ABP subcutaneously had endogenous VWF levels being increased 3-4 fold. These results are compatible with KB-VWF-013bv_ABP being able to increase VWF antigen levels over a prolonged period of time, irrespective whether the fusion-protein is given intravenously or subcutaneously. Data represent mean±SD of 3-12 mice (panel A and open circles in panel B). Closed circles in panel B represent mean±range of 2 mice.

FIG. 7: Increasing endogenous FVIII activity levels via injection of KB-VWF-013bv_ABP. FVIII activity levels were measured in a subset of the samples obtained from the experiment the results of which are depicted in FIG. 6: 4 mice that received KB-VWF-013bv_ABP (2.5 mg/kg bodyweight), in 3 mice that received vehicle, and two mice that received KB-VWF-103bv_ABP subcutaneously (2.5 mg/kg bodyweight). FVIII activity levels measured in pre-injection samples were considered as FVIII activity levels at t=0, and were arbitrarily set at 100%. In contrast to mice treated with vehicle, mice injected intravenously with KB-VWF-013bv_ABP are characterized by a >5-fold increase in FVIII activity levels when measured three days after injection. Both mice receiving KB-VWF-013bv_ABP subcutaneously had endogenous FVIII activity levels being increased 2-5 fold. These results are compatible with KB-VWF-013bv_ABP being able to increase FVIII activity levels over a prolonged period of time, irrespective whether the fusion-protein is given intravenously or subcutaneously. Data represent mean±SD of 3-8 mice (open and closed circles in panel A & open circles in panel B). Closed circles in panel B represent mean±range of 2 mice.

### EXAMPLES

Example 1: Binding of KB-AT-002/003_ABP to human and murine albumin in an immunosorbent assay. Binding to albumin may be favorable to increase the size of sdAbs, which are smaller sized than conventional immunoglobulins (15 kDa versus 150 kDa, for a monovalent sdAb and IgG, respectively). A construct was established encoding a bi-paratopic sdAb (KB-AT-002/003) fused to an albumin binding peptide (ABP, with the amino acid sequence QGLIGDIALPRWGALWGDSV) resulting in the sdAb designated KB-AT-002/003_ABP (SEQ ID NO: 18). Purified KB-AT-002/003_ABP was tested for binding to both human and murine albumin. Human and murine albumin were immobilized (10 microgram/ml) in 10 mM NaHCO₃, 50 mM Na2CO3 (pH 9.5) in a volume of 50 microliter in microtiter plates (Greiner Bio-One, Les Ulis, France) for 16 h at 4° C. As a negative control, no albumin was immobilized. After washing the wells three times with 100 microliter/well using Tris-buffered saline (pH 7.6) supplemented with 0.1% Tween-20 (TBS-T), various concentrations of KB-AT-002/003_ABP (0-10 microgram/ml; in TBS-T, 50 μl per well, 2 hours at 37° C.) were added to albumin-coated wells and non-coated control wells.

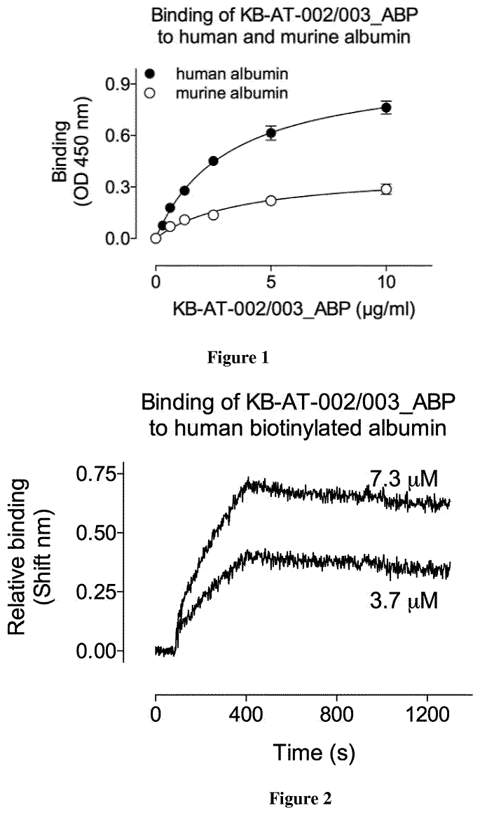

Wells were then washed three times with 200 microliter/well using TBS-T. Bound of KB-AT-002/003_ABP was probed with peroxidase-labeled monoclonal anti-His tag antibodies (Abcam, diluted 1/10000) for 2 hours at 37° C. with 50 microliter/well. Wells were then washed three times with 100 microliter/well using TBS-T. Residual peroxidase activity was detected by measuring peroxidase-mediated hydrolysis of 3,3′,5,5′-tetramethylbenzidine. Plotted in FIG. 1 is the binding of KB-AT-002/003_ABP to both human and murine albumin, corrected for binding to non-coated wells. KB-AT-002/003_ABP displays a saturable and dose-dependent binding to both human and murine albumin. This shows that fusion of KB-AT-002/003_ABP induces association of the sdAB to human and murine albumin. KB-AT-002/003 without the albumin binding peptide did not bind to human or murine albumin under these conditions.

Example 2: Binding of KB-AT-002/003_ABP to biotinylated human albumin in a biolayer-interferometry analysis assay. We tested the binding of KB-AT-002/003_ABP to biotinylated human albumin using Octet-QK equipment (FIG. 2). To this end, biotinylated albumin (bt-albumin) was diluted in phosphate-buffered saline with 0.1% Tween-20 (PBS-T) to a concentration of 200 microgram/ml for absorption to streptavidin-high binding capacity sensors (Fortébio, Menlo Park, Calif., USA). Sensors were rehydrated in 0.2 ml PBS-T for 15 min. Sensors were then incubated with 0.1 ml bt-albumin solution for 600 sec. Sensors were subsequently allowed to reach stable baseline levels via incubation with PBS-T for 300 sec. bt-Albumin coated sensors were then transferred to wells containing KB-AT-002/003_ABP (125 or 250 microgram/ml in PBS-T) and incubated for 300 sec in order to visualize association of KB-AT-002/003_ABP to immobilized bt-albumin. Following this association phase, sensors were transferred to wells containing PBS-T, and incubated for 900 sec, allowing dissociation of the KB-AT-002/003_ABP/bt-albumin complex. As is shown in FIG. 2, KB-AT-002/003_ABP displayed binding to human bt-albumin in a dose-dependent and reversible manner. Analysis of the association- and dissociation curves allowed calculating a preliminary apparent affinity constant of 0.42±0.02 micromolar.

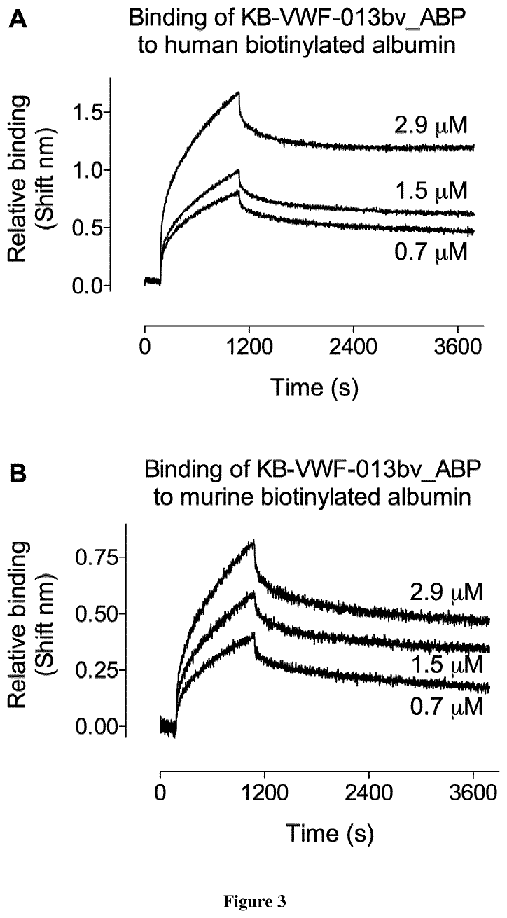

Example 3: Binding of KB-VWF-013bv_ABP to biotinylated human or murine albumin in a biolayer-interferometry analysis assay. We tested the binding of KB-VWF-013bv_ABP (SEQ ID NO: 6) to biotinylated human or murine albumin using Octet-QK equipment (FIG. 3). To this end, biotinylated albumin (bt-albumin) was diluted in phosphate-buffered saline with 0.1% Tween-20 (PBS-T) to a concentration of 200 microgram/ml for absorption to streptavidin-high binding capacity sensors (Fortébio, Menlo Park, Calif., USA). Sensors were rehydrated in 0.2 ml PBS-T for 15 min. Sensors were then incubated with 0.1 ml bt-albumin solution for 600 sec. Sensors were subsequently allowed to reach stable baseline levels via incubation with PBS-T for 300 sec. bt-Albumin coated sensors were then transferred to wells containing KB-VWF-013bv_ABP (125 or 250 microgram/ml in PBS-T) and incubated for 900 sec in order to visualize association of KB-VWF-013bv_ABP to immobilized bt-albumin. Following this association phase, sensors were transferred to wells containing PBS-T, and incubated for 2700 sec, allowing dissociation of the KB-VWF-013_ABP/bt-albumin complex. As is shown in FIG. 3, KB-VWF-013bv_ABP displayed binding to both human and murine bt-albumin in a dose-dependent and reversible manner. Analysis of the respective association- and dissociation curves allowed calculating preliminary apparent affinity constants of 99±65 nM and 83±34 nM for human and murine bt-albumin respectively.

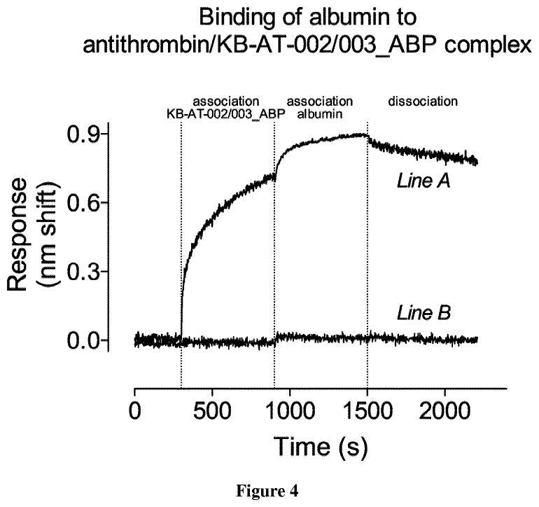

Example 4: Binding of murine albumin to a complex of antithrombin and KB-AT-002/003_ABP. We tested the binding of albumin to KB-AT-002/003_ABP while the sdAb was bound to antithrombin via biolayer-interferometry analysis using Octet-QK equipment (FIG. 4). To this end, human antithrombin was diluted in 0.1 M Mes (pH 5.0) to a concentration of 50 microgram/ml for coupling to EDC/NHS-activated amine-reactive biosensors (Fortébio, Menlo Park, Calif., USA). Sensors were rehydrated in 0.2 ml 0.1 M MES, pH 5.0 for 300 sec. Sensors were then activated via incubation with 0.1 ml 0.2 M EDC/0.095 M NHS mixture for 300 sec and subsequently incubated with 0.1 ml antithrombin-solution for 600 sec. Unoccupied amine-reactive sites were quenched by incubating with 1M ethanolamine for 180 sec, and sensors were allowed to reach stable baseline levels via incubation with phosphate-buffered saline supplemented with 0.1% Tween-20 (PBS-T) for 300 sec. Antithrombin-coated sensors were then transferred to wells containing KB-AT-002/003_ABP (100 microgram/ml in PBS-T) and incubated for 600 sec in order to visualize association of KB-AT-002/003_ABP to immobilized antithrombin. Following this association phase, sensors were transferred to wells containing murine albumin (0.5 mg/ml in PBS-T) and incubated for 600 sec, allowing association of albumin to the antithrombin/KB-AT-002/003_ABP complex. Finally, sensors were transferred to wells containing PBS-T and incubated for 900 sec, allowing dissociation of the antithrombin/KB-AT-002/003_ABP/albumin complex. As is showing in the lower panel of FIG. 4, KB-AT-002/003_ABP bound readily to immobilized antithrombin. Subsequently, murine albumin associated to the antithrombin/KB-AT-002/003_ABP complex, while little dissociation of the antithrombin/KB-AT-002/003_ABP/albumin complex was observed in the final stage of the experiment. Of note, no albumin binding was observed to complexes consisting of antithrombin/KB-AT-002/003 (which lacks the albumin binding peptide), indicating that the observed association of albumin is specific for the presence of the albumin binding peptide.

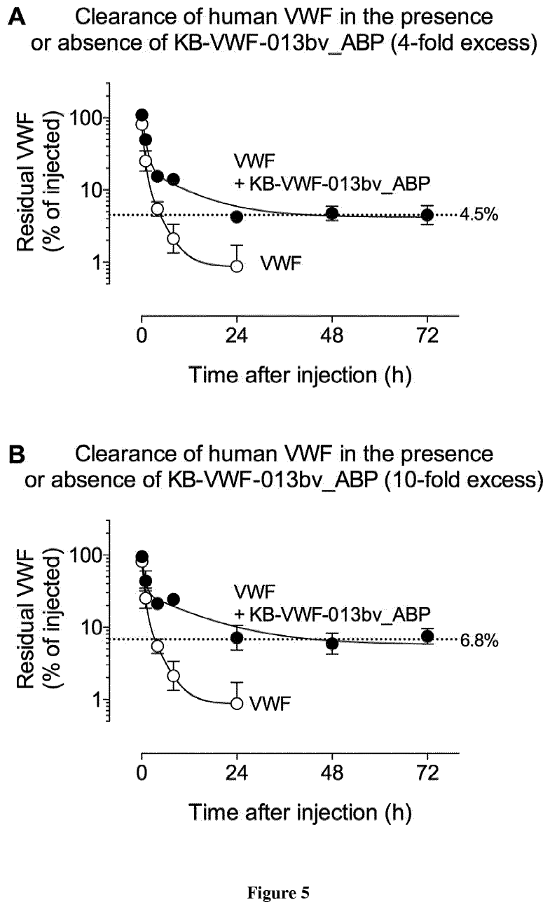

Example 5: Clearance of von Willebrand factor in the presence or absence of KB-VWF-013bv_ABP. Purified plasma-derived human von Willebrand factor (VWF; 25 microgram/ml) was incubated in the absence or presence of KB-VWF-013bv_ABP (SEQ ID NO: 6; 13.5 microgram/ml [4-fold molar excess] for 15 min or 33.8 microgram/ml [10-fold molar excess] for 30 min) in PBS. Subsequently, the solution was given to wild-type C57B6/J-mice (0.25 mg VWF/kg bodyweight; 0.63 mg/kg or 1.7 mg/kg KB-VWF-013bv_ABP/kg bodyweight) via intravenous tail injection. At different time-points after injection (3 min, 1 h, 4 h, 8 h, 24 h, 48 h, 72 h) blood samples were obtained via retro-orbital puncture from isoflurane-anesthetized mice and citrated plasma was prepared by centrifugation (1500 g for 20 min at 22° C.). For each time point, 3-18 mice were analyzed. Mice were generally bled once or twice. In some cases, mice were bled three of four times with time intervals of >20 h. Residual plasma concentrations of human VWF were determined employing an in-house ELISA that specifically measures human VWF, employing a pool of murine monoclonal anti-VWF antibodies as capturing agent and peroxidase-labeled polyclonal rabbit anti-VWF antibodies (Dakocytomation, Glostrup, Denmark) as probing agent. Recoveries at 3 min after injection were 4.1±0.7 microgram/ml (n=18), 5.5±0.4 microgram/ml (n=12 for 0.63 mg/kg; p<0.0001 in one-way Anova with Dunnet's correction for multiple comparisons) and 4.8±0.9 microgram/ml (n=12 for 1.7 mg/kg; p=0.0159) for VWF injected in the absence or presence of KB-VWF-013bv_ABP, respectively. This suggests that the initial recovery is higher for VWF injected in the presence of KB-VWF-013bv_ABP compared to VWF injected in its absence. We then plotted residual VWF antigen concentrations relative to those at 3 min after injection versus time after injection (FIGS. 5A and 5B). At all time-points, VWF levels were consistently higher in mice injected with VWF in the presence of KB-VWF-013bv_ABP compared to mice injected with VWF alone.

TABLE 1
Residual VWF antigen levels following
intravenous injection in the absence or
presence of KB-VWF-013bv_ABP.

| Time | | VWF + | VWF + |
|---|---|---|---|
| after | VWF | KB-VWF- | KB-VWF- |
| injection | (% of | 013bv_ABP* | 013bv_ABP** |
| (h) | injected) | (% of injected) | (% of injected) |
| 0.05 | 81 ± 13 | 109 ± 16 | 95 ± 18 |
| 1 | 25 ± 10 | 46 ± 2 | 44 ± 16 |
| 4 | 5.5 ± 1.4 | 14 ± 2 | 21 ± 2 |
| 8 | 2.1 ± 1.2 | 13 ± 1 | 24 ± 2 |
| 24 | 0.9 ± 0.8 | 3.9 ± 1.0 | 7.1 ± 3.5 |
| 48 | ND | 4.8 ± 1.2 | 5.9 ± 2.4 |
| 72 | ND | 4.4 ± 1.6 | 7.5 ± 2.1 |

ND: not detectable; data are mean ± SD; n = 3-18 per time point. *0.63 mg/kg; **1.7 mg/kg
Moreover, no VWF could be detected at time-points 48 h and 72 h when injected in the absence of KB-VWF-013bv_ABP. In contrast, substantial concentrations of VWF could still be detected at these time-points following injection in the presence of KB-VWF-013bv_ABP, irrespective whether this was given at a dose of 0.63 mg/kg or 1.7 mg/kg. These levels remained more or less stable over the period 24-72 hours after injection. These data demonstrate that associating VWF with the anti-VWF sdAb KB-VWF-013bv fused to the ABP considerably increases the circulatory half-life of VWF.

Example 6: Intravenous or subcutaneous injection of KB-VWF-013bv_ABP results in sustained increased levels of endogenous VWF. We have tested the ability of KB-VWF-013bv_ABP to raise endogenous levels of VWF in wild-type mice. Therefore, wild-type C57B6/J-mice were given a single intravenous dose (2.5 mg/kg bodyweight) of KB-VWF-013bv_ABP or vehicle. Two mice were given a single subcutaneous (2.5 mg/kg) of KB-VWF-013bv_ABP. Twenty-four hours before injection and at different time-points after injection (1 day, 3 days, 7 days and 12 days) blood samples were obtained via retro-orbital puncture from isoflurane-anesthetized mice and citrated plasma was prepared by centrifugation (1500 g for 20 min at 22° C.). For each time point, 3-12 mice were analyzed. Plasma concentrations of endogenous murine VWF were determined employing an in-house ELISA that employs polyclonal rabbit anti-VWF antibodies as capturing agent and peroxidase-labeled polyclonal rabbit anti-VWF antibodies (both from Dakocytomation, Glostrup, Denmark) as probing agent.

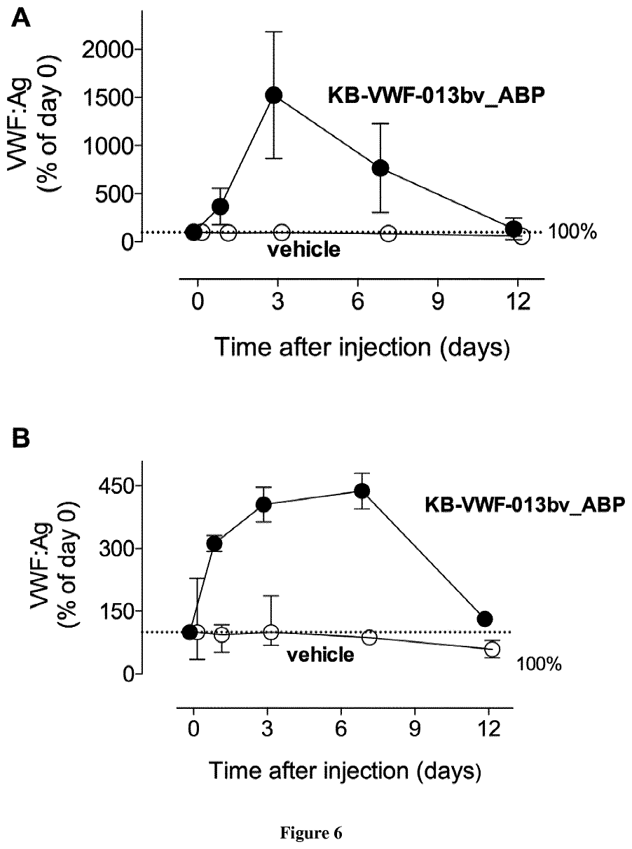

Normal mouse plasma was used as reference. At base-line levels (i.e. VWF antigen levels in plasma from mice before injection), VWF levels were similar in mice to be injected with vehicle (89±57% of normal mouse plasma; n=8) compared to mice receiving KB-VWF-013bv_ABP intravenously (66±16%; n=12) or subcutaneously (64% and 56%). In the vehicle-treated group, no increase in VWF levels was detected over the observation period of 12 days (FIGS. 6A and 6B). In contrast, VWF levels were increased at days 1 (3.7±1.9 fold (n=4; p=0.06 compared to vehicle-treated mice in multiple t-test comparison), at day 3 (15.2±6.6 fold; n=12; p<1.0×10-5 compared to vehicle-treated mice in multiple t-test comparison) and at day 7 (7.7±4.6 fold; n=12; p=0.0006 compared to vehicle-treated mice in multiple t-test comparison) (FIG. 6A). VWF antigen levels were returned to near baseline levels at day 12 (increase=1.4±1.1 fold compared to day 0 value). As for the two mice receiving a subcutaneous injection with KB-VWF-013bv_ABP, VWF levels were increased 3-4-fold at days 1, 3 and 7 after injection (3.3-& 2.9-fold, 3.6- & 4.5-fold and 3.9- & 4.8-fold, respectively; FIG. 6B). Apparently, a single bolus injection of KB-VWF-013bv_ABP, either intravenously or subcutaneously, results in a sustained increase of endogenous VWF levels.

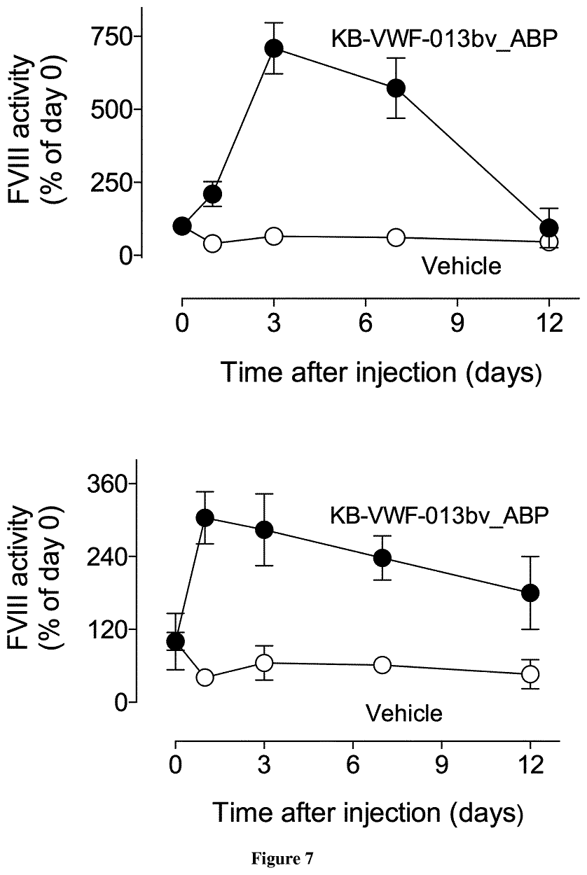

Example 7: Intravenous or subcutaneous injection of KB-VWF-013bv_ABP results in sustained increased levels of endogenous Factor VIII. Because VWF circulates in complex with factor VIII (FVIII), we analyzed in a subset of samples described in example 6 whether the injection of KB-VWF-013bv_ABP was associated with a concomitant rise in FVIII activity levels (FIG. 7). FVIII levels were measured in 3-8 mice that received KB-VWF-013bv_ABP (2.5 mg/kg bodyweight) intravenously, in 3-6 mice that received vehicle intravenously, and in two mice that received KB-VWF-103bv_ABP subcutaneously (2.5 mg/kg bodyweight). FVIII levels were measured in a chromogenic FVIII activity test (Biophen FVIII:C; Hyphen Biomed, Neuville-sur-Oise, France), while normal mouse plasma was used as reference. FVIII activity levels obtained 24 hours before injection were arbitrarily set at 100% and considered as values at day 0, and used as comparison for levels measured in samples taken at day 1, day 3, day 7 and day 12 after injection. No statistical significant differences in FVIII levels at day 0 were detected between the three different groups. In the vehicle-treated mice, no increase in FVIII levels was observed during the 12-day observation period (FIG. 7A and FIG. 7B). In mice that received KB-VWF-013bv_ABP intravenously, FVIII levels were increased 2.1±0.9-fold at day 1, 7.1±2.5-fold at day 3, 5.7±2.7-fold at day 7 (FIG. 7A). At day 12, FVIII levels had returned to baseline values. As for the two mice receiving a subcutaneous injection with KB-VWF-013bv_ABP, FVIII activity levels were increased 2-3-fold at days 1, 3, and 7 after injection (2.7- & 3.3-fold, 3.3- & 2.4-fold and 2.1- & 2.6-fold, respectively) (FIG. 7B). At day 12, FVIII levels were increased 2.2- and 1.4-fold. Apparently, a single bolus injection of KB-VWF-013bv_ABP, either intravenously or subcutaneously, results in a sustained increase of endogenous FVIII activity levels.

### REFERENCES

Throughout this application, various references describe the state of the art to which this invention pertains. The disclosures of these references are hereby incorporated by reference into the present disclosure.

1. A peptide comprising the amino acid sequence QGLIGDIALPRWGALWGDSV (SEQ ID NO: 1).

2. A nucleic acid encoding an amino acid sequence comprising SEQ ID NO: 1.

3. An expression vector comprising the nucleic acid according to claim 2.

4. A host cell comprising the expression vector according to claim 3.

5. A drug conjugate comprising the peptide according to claim 1 linked to a heterologous moiety.

6. The drug conjugate according to claim 17 wherein the heterologous polypeptide is an isolated single domain antibody.

7. The drug conjugate according to claim 1, wherein the peptide of claim 1 is fused to an isolated single domain antibody to form a fusion protein.

8. The drug conjugate according to claim 6, wherein the isolated single domain antibody is directed against at least one endogenous protein selected from the group consisting of: Von Willebrand factor, Fibrinogen, Factor II (prothrombin), Factor V, Factor VII, Factor VIII, Factor IX, Factor X, Factor XI, Factor XII, Factor XIII, Protein C, Protein S, Protein Z, Protein Z-inhibitor, Tissue factor pathway inhibitor (TFPI), α1-antitrypsin inhibitor, Thrombin Activatable Fibrinolysis Inhibitor (TAFI)/carboxypeptidase B2, Antithrombin, α2-antiplasmin, Plasmin Activator Inhibitor-1 (PAI-1), Plasminogen, tissue plasminogen activator (tPA), urinary plasminogen activator (uPA), ADAMTS13, Complement protein C2, Complement protein C3, Complement protein C4, Complement protein C6, Complement factor H, Complement factor 1, Properdin, Ceruloplasmin, Kininogen, Thrombopoietin, Erytropoeitin, Soluble Mannose-binding lectin, Interferon-α, Interferon-β, Interferon-γ, Granulocyte colony-stimulating factor, Granulocyte-macrophage colony-stimulating factor, Keratinocyte growth factor, Interleukin-2, Interleukin-6, Interleukin-7, Interleukin-10, Interleukin-11, Interleukin-12, Interleukin-15, Interleukin-21, Growth hormone-releasing hormone and Hyaluronidase.

9. The drug conjugate according to claim 6, wherein the isolated single domain antibody comprises:
i) a CDR1 having at least 70% of identity with a sequence set forth as SEQ ID NO:2, a CDR2 having at least 70% of identity with a sequence set forth as SEQ ID NO:3 and a CDR3 having at least 70% of identity with a sequence set forth as SEQ ID NO:4;
ii) a CDR1 having at least 70% of identity with a sequence set forth as SEQ ID NO:7, a CDR2 having at least 70% of identity with a sequence set forth as SEQ ID NO:8 and a CDR3 having at least 70% of identity with a sequence set forth as SEQ ID NO:9;
iii) a CDR1 having at least 70% of identity with a sequence set forth as SEQ ID NO:12, a CDR2 having at least 700/of identity with a sequence set forth as SEQ ID NO:13 and a CDR3 having at least 70/o of identity with a sequence set forth as SEQ ID NO:14;
iv) a CDR1 having at least 70% of identity with a sequence set forth as SEQ ID NO:17, a CDR2 having at least 70% of identity with a sequence set forth as SEQ ID NO:18 and a CDR3 having at least 70% of identity with a sequence set forth as SEQ ID NO:19;
v) a CDR1 having at least 70% of identity with a sequence set forth as SEQ ID NO:21, a CDR2 having at least 70% of identity with a sequence set forth as SEQ ID NO:22 and a CDR3 having at least 70% of identity with a sequence set forth as SEQ ID NO:23;
vi) a CDR1 having at least 70% of identity with a sequence set forth as SEQ ID NO:25, a CDR2 having at least 70% of identity with a sequence set forth as SEQ ID NO:26 and a CDR3 having at least 70% of identity with a sequence set forth as SEQ ID NO:27;
vii) a CDR1 having at least 70% of identity with a sequence set forth as SEQ ID NO:29, a CDR2 having at least 70% of identity with a sequence set forth as SEQ ID NO:30 and a CDR3 having at least 70% of identity with a sequence set forth as SEQ ID NO:31;
viii) a CDR1 having at least 70% of identity with a sequence set forth as SEQ ID NO:33, a CDR2 having at least 70% of identity with a sequence set forth as SEQ ID NO:34 and a CDR3 having at least 70% of identity with a sequence set forth as SEQ ID NO:35;
ix) a CDR1 having at least 70% of identity with a sequence set forth as SEQ ID NO:37, a CDR2 having at least 70% of identity with a sequence set forth as SEQ ID NO:38 and a CDR3 having at least 70% of identity with a sequence set forth as SEQ ID NO:39;
x) a CDR1 having at least 70% of identity with a sequence set forth as SEQ ID NO:41, a CDR2 having at least 70% of identity with a sequence set forth as SEQ ID NO:42 and a CDR3 having at least 70% of identity with a sequence set forth as SEQ ID NO:43;
xi) a CDR1 having at least 70% of identity with a sequence set forth as SEQ ID NO:54, a CDR2 having at least 70% of identity with a sequence set forth as SEQ ID NO:55 and a CDR3 having at least 70% of identity with a sequence set forth as SEQ ID NO:56;
xii) a CDR1 having at least 70% of identity with a sequence set forth as SEQ ID NO:59, a CDR2 having at least 70%/0 of identity with a sequence set forth as SEQ ID NO:60 and a CDR3 having at least 70% of identity with a sequence set forth as SEQ ID NO:61:
xiii) a CDR1 having at least 70% of identity with a sequence set forth as SEQ ID NO:64, a CDR2 having at least 70% of identity with a sequence set forth as SEQ ID NO:65 and a CDR3 having at least 70% of identity with a sequence set forth as SEQ ID NO:66;
xiv) a CDR1 having at least 70% of identity with a sequence set forth as SEQ ID NO:69, a CDR2 having at least 70% of identity with a sequence set forth as SEQ ID NO:70 and a CDR3 having at least 70% of identity with a sequence set forth as SEQ ID NO:71;
xv) a CDR1 having at least 70% of identity with a sequence set forth as SEQ ID NO:74, a CDR2 having at least 70% of identity with a sequence set forth as SEQ ID NO:75 and a CDR3 having at least 70% of identity with a sequence set forth as SEQ ID NO:76;
xvi) a CDR1 having at least 70% of identity with 1 sequence set forth as SEQ ID NO:79, a CDR2 having at least 70% of identity with a sequence set forth as SEQ ID NO:80 and a CDR3 having at least 70% of identity with a sequence set forth as SEQ ID NO:81;
xvii) a CDR1 having at least 70% of identity with a sequence set forth as SEQ ID NO:84, a CDR2 having at least 70% of identity with a sequence set forth as SEQ ID NO:85 and a CDR3 having at least 70% of identity with a sequence set forth as SEQ ID NO:86; or
xviii) a CDR1 having at least 70% of identity with a sequence set forth as SEQ ID NO:89, a CDR2 having at least 70% of identity with a sequence set forth as SEQ ID NO:90 and a CDR3 having at least 70% of identity with a sequence set forth as SEQ ID NO:91.

10. The drug conjugate according to claim 6, wherein the isolated single domain antibody comprises:
i) a CDR1 having a sequence set forth as SEQ ID NO:2, a CDR2 having a sequence set forth as SEQ ID NO:3 and a CDR3 having a sequence set forth as SEQ ID NO:4;
ii) a CDR1 having a sequence set forth as SEQ ID NO:7, a CDR2 having a sequence set forth as SEQ ID NO:8 and a CDR3 having a sequence set forth as SEQ ID NO:9;
iii) a CDR1 having a sequence set forth as SEQ ID NO:12, a CDR2 having a sequence set forth as SEQ ID NO:13 and a CDR3 having a sequence set forth as SEQ ID NO:14;
iv) a CDR1 having a sequence set forth as SEQ ID NO:17, a CDR2 having a sequence set forth as SEQ ID NO:18 and a CDR3 a sequence set forth as SEQ ID NO:19;
v) a CDR1 having a sequence set forth as SEQ ID NO:21, a CDR2 having g sequence set forth as SEQ ID NO:22 and a CDR3 having a sequence set forth as SEQ ID NO:23;
vi) a CDR1 having a sequence set forth as SEQ ID NO:25, a CDR2 having a sequence set forth as SEQ ID NO:26 and a CDR3 having a sequence set forth as SEQ ID NO:27;
vii) a CDR1 having a sequence set forth as SEQ ID NO:29, a CDR2 having a sequence set forth as SEQ ID NO:30 and a CDR3 having a sequence set forth as SEQ ID NO:31;
viii) a CDR1 having a sequence set forth as SEQ ID NO:33, a CDR2 having a sequence set forth as SEQ ID NO:34 and a CDR3 having a sequence set forth as SEQ ID NO:35;
ix) a CDR1 having a sequence set forth as SEQ ID NO:37, a CDR2 having a sequence set forth as SEQ ID NO:38 and a CDR3 having a sequence set forth as SEQ ID NO:39;
x) a CDR1 having a sequence set forth as SEQ ID NO:41, a CDR2 having a sequence set forth as SEQ ID NO:42 and a CDR3 having a sequence set forth as SEQ ID NO:43;
xi) a CDR1 having a sequence set forth as SEQ ID NO:54, a CDR2 having a sequence set forth as SEQ ID NO:55 and a CDR3 having a sequence set forth as SEQ ID NO:56;
xii) a CDR1 having a sequence set forth as SEQ ID NO:59, a CDR2 having a sequence set forth as SEQ ID NO:60 and a CDR3 having a sequence set forth as SEQ ID NO:61;
xiii) a CDR1 having a sequence set forth as SEQ ID NO:64, a CDR2 having a sequence set forth as SEQ ID NO:65 and a CDR3 having a sequence set forth as SEQ ID NO:66;
xiv) a CDR1 having a sequence set forth as SEQ ID NO:69, a CDR2 having a sequence set forth as SEQ ID NO:70 and a CDR3 having a sequence set forth as SEQ ID NO:71;
xv) a CDR1 having a sequence set forth as SEQ ID NO:74, a CDR2 having a sequence set forth as SEQ ID NO:75 and a CDR3 having a sequence set forth as SEQ ID NO:76;
xvi) a CDR1 having a sequence set forth as SEQ ID NO:79, a CDR2 having a sequence set forth as SEQ ID NO:80 and a CDR3 having a sequence set forth as SEQ ID NO:81;
xvii) a CDR1 having a sequence set forth as SEQ ID NO:84, a CDR2 having g sequence set forth as SEQ ID NO:85 and a CDR3 having a sequence set forth as SEQ ID NO:86; or
xviii) a CDR1 having a sequence set forth as SEQ ID NO:89, a CDR2 having a sequence set forth as SEQ ID NO:90 and a CDR3 having a sequence set forth as SEQ ID NO:91.

11. The drug conjugate according to, claim 6, wherein the isolated single domain antibody is:
i) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO:5;
ii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO:10;
iii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO:15;
iv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 20;
v) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 24;
vi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 28;
vii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 32;
viii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 36;
ix) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 40;
x) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 44;
xi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 57;
xii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO; 62;
xiii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 67;
xiv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEO ID NO: 72;
xv) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 77;
xvi) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 82;
xvii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, %, 97, 98, 99 or 100% of identity with the sequence set forth as SEQ ID NO: 87; or
xviii) an isolated single domain antibody having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of identity with the sequence set forth as SEQ ID NO: 92.

12. The drug conjugate according to claim 6, wherein the isolated single domain antibody is or comprises:
i) an isolated single domain antibody having a sequence set forth as SEQ ID NO:5;
ii) an isolated single domain antibody having a sequence set forth as SEQ ID NO: 10;
iii) an isolated single domain antibody having a sequence set forth as SEQ ID NO: 15;
iv) an isolated single domain antibody having a sequence set forth as SEQ ID NO: 20
v) an isolated single domain antibody having a sequence set forth as SEQ ID NO:24;
vi) an isolated single domain antibody having a sequence set forth as SEQ ID NO:28;
vii) an isolated single domain antibody having a sequence set forth as SEQ ID NO: 32;
viii) an isolated single domain antibody having a sequence set forth as SEQ ID NO: 36;
ix) an isolated single domain antibody having a sequence set forth as SEQ ID NO: 40;
x) an isolated single domain antibody having a sequence set forth as SEQ ID NO: 44;
xi) an isolated single domain antibody having a sequence set forth as SEQ ID NO: 57;
xii) an isolated single domain antibody having a sequence set forth as SEQ ID NO: 62;
xiii) an isolated single domain antibody having a sequence set forth as SEQ ID NO: 67;
xiv) an isolated single domain antibody having a sequence set forth as SEQ ID NO: 72;
xv) an isolated single domain antibody having a sequence set forth as SEQ ID NO: 77;
xvi) an isolated single domain antibody having a sequence set forth as SEQ ID NO: 82;
xvii) an isolated single domain antibody having a sequence set forth as SEQ ID NO: 87; or
xviii) an isolated single domain antibody having a sequence set forth as SEQ ID NO: 92.

13. The drug conjugate according to claim 6, wherein said isolated single domain antibody is fused to a peptide comprising the amino acid sequence OGLIGDIALPRWGALWGDSV (SEQ ID NO: 1) and a sequence set forth as SEQ ID NO: 6; SEQ ID NO: 11; SEQ ID NO: 16; SEQ ID NO: 49; SEQ ID NO: 58, SEQ ID NO: 63, SEQ ID NO: 68, SEQ ID NO: 73: SEQ ID NO: 78; SEQ ID NO: 83, SEQ ID NO: 88 or SEQ ID NO: 93.

14. The drug conjugate according to claim 6, wherein the isolated single domain antibody comprises: i) the sequences KB-AT-002 and one KB-AT-003 having at least 70% sequence identity with a sequence set forth as SEQ ID NO: 45; ii) the sequences KB-AT-001 and KB-AT-002 having at least 70% sequence identity with a sequence set forth as SEQ ID NO: 46; iii) the sequences KB-AT-001 and KB-AT-003 having at least 70% sequence identity with a sequence set forth as SEQ ID NO: 47; iv) the sequences KB-AT-001 and KB-AT-005 having at least 70% sequence identity with a sequence set forth as SEQ ID NO: 48; v) the two sequences of KB-AT-001 and one sequence of KB-AT-002, having at least 70% sequence identity with a sequence set forth as SEQ ID NO: 50; vi) the two sequences of KB-AT-001 and KB-AT-003 having at least 70% sequence identity with a sequence set forth as SEQ ID NO: 51; vii) the two sequences of KB-AT-001 and KB-AT-005 having at least 70% sequence identity with a sequence set forth as SEQ ID NO: 52 or two sequences of KB-AT-001, one of KB-AT-002 sequence and one sequence of KB-AT-003, having at least 70% sequence identity with a sequence set forth as SEQ ID NO: 53.

15. A method of extending or increasing the half-life and the level of an endogenous protein in a subject comprising a step of administering to the subject the drug conjugate according to claim 5 wherein the drug conjugate is or is not inserted into a vector.

16. A method of treating a subject in need thereof comprising a step of administering to said subject a therapeutically effective amount of the drug conjugate according to claim 5.

17. The drug conjugate of claim 5, wherein the heterologous moiety is a heterologous polypeptide.


SEQUENCE LISTING (94 sequences)

SEQ ID NO: 1 - protein, 20 aa, Artificial
  Synthetic SEQ ID NO 1
QGLIGDIALPRWGALWGDSV

SEQ ID NO: 2 - protein, 10 aa, Artificial
  Synthetic KB-VWF-013 CDR1 SEQ ID NO 2
GRTFIRYAMA

SEQ ID NO: 3 - protein, 16 aa, Artificial
  Synthetic  KB-VWF-013 CDR2 SEQ ID NO 3
IPQSGGRSYYADSVKG

SEQ ID NO: 4 - protein, 19 aa, Artificial
  Synthetic KB-VWF-013 CDR3 SEQ ID NO 4
TSTYYGRSAYSSHSGGYDY

SEQ ID NO: 5 - protein, 128 aa, Artificial
  Synthetic KB-VWF-013 SEQ ID NO 5
QVQLVQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSS

SEQ ID NO: 6 - protein, 309 aa, Artificial
  Synthetic KB-VWF-013bv-ABP SEQ ID NO 6
QVQLQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSSGGGSGGGSGGGSGGGSQVQLVQSGGGLVQAGDSLRLSCAASGRTFIRYAMAWFRQAPGKEREFVAAIPQSGGRSYYADSVKGRFTISRDNAKNTVYLQMNSLKPEDTAVYSCAATSTYYGRSAYSSHSGGYDYWGQGTQVTVSSGRGGGSLTPRGVRLGGGSQGLIGDIALPRWGALWGDSV

SEQ ID NO: 7 - protein, 10 aa, Artificial
  Synthetic KB-VWF-008 CDR1 SEQ ID NO 7
GRTFSDYAMG

SEQ ID NO: 8 - protein, 16 aa, Artificial
  Synthetic KB-VWF-008 CDR2 SEQ ID NO 8
INRSGGRLSYAESVND

SEQ ID NO: 9 - protein, 16 aa, Artificial
  Synthetic KB-VWF-008 CDR3 SEQ ID NO 9
RTNWNPPRPLPEEYNY

SEQ ID NO: 10 - protein, 125 aa, Artificial
  Synthetic KB-VWF-008 SEQ ID NO 10
QVQLVQSGGGLVQAGDSLKLSCAASGRTFSDYAMGCILQNPGKERDFVASINRSGGRLSYAESVNDLFTISVDNAKNMLYLQMNSLKPEDTAVHYCVLRTNWNPPRPLPEEYNYWGQETQVTVSS

SEQ ID NO: 11 - protein, 304 aa, Artificial
  Synthetic KB-VWF-008bv-ABP SEQ ID NO 11
QVQLVQSGGGLVQAGDSLKLSCAASGRTFSDYAMGCILQNPGKERDFVASINRSGGRLSYAESVNDLFTISVDNAKNMLYLQMNSLKPEDTAVHYCVLRTNWNPPRPLPEEYNYWGQETQVTVSSGGGSGGGSGGGSGGGSQVQLVQSGGGLVQAGDSLKLSCAASGRTFSDYAMGCILQNPGKERDFVASINRSGGRLSYAESVNDLFTISVDNAKNMLYLQMNSLKPEDTAVHYCVLRTNWNPPRPLPEEYNYWGQETQVTVSSGRGGGSLTPRGVRLGGGSQGLIGDIALPRWGALWGDSV

SEQ ID NO: 12 - protein, 10 aa, Artificial
  Synthetic KB-VWF-011 CDR1 SEQ ID NO 12
GGTFSNYAMG

SEQ ID NO: 13 - protein, 16 aa, Artificial
  Synthetic KB-VWF-011 CDR2 SEQ ID NO 13
ISRSGHRTDYADSAKG

SEQ ID NO: 14 - protein, 15 aa, Artificial
  Synthetic KB-VWF-011 CDR3 SEQ ID NO 14
RSDWSIATTATSYDY

SEQ ID NO: 15 - protein, 124 aa, Artificial
  Synthetic KB-VWF-011 SEQ ID NO 15
QVQLVQSGGGLVQAGDSLRLSCAASGGTFSNYAMGWFRQTPGKEREFVARISRSGHRTDYADSAKGRFTISRDNAKNTVYLQMNSLKPEDTAVYYCAARSDWSIATTATSYDYWGQGTQVTVSS

SEQ ID NO: 16 - protein, 302 aa, Artificial
  Synthetic KB-VWF-011bv-ABP SEQ ID NO 16
QVQLVQSGGGLVQAGDSLRLSCAASGGTFSNYAMGWFRQTPGKEREFVARISRSGHRTDYADSAKGRFTISRDNAKNTVYLQMNSLKPEDTAVYYCAARSDWSIATTATSYDYWGQGTQVTVSSGGGSGGGSGGGSGGGSQVQLVQSGGGLVQAGDSLRLSCAASGGTFSNYAMGWFRQTPGKEREFVARISRSGHRTDYADSAKGRFTISRDNAKNTVYLQMNSLKPEDTAVYYCAARSDWSIATTATSYDYWGQGTQVTVSSGRGGGSLTPRGVRLGGGSQGLIGDIALPRWGALWGDSV

SEQ ID NO: 17 - protein, 8 aa, Artificial
  Synthetic KB-AT-001 CDR1 SEQ ID NO 17
GRTFRNYV

SEQ ID NO: 18 - protein, 8 aa, Artificial
  Synthetic KB-AT-001 CDR2 SEQ ID NO 18
INRSGAIT

SEQ ID NO: 19 - protein, 16 aa, Artificial
  Synthetic KB-AT-001 CDR3 SEQ ID NO 19
AAGETTWSIRRDDYDY

SEQ ID NO: 20 - protein, 123 aa, Artificial
  Synthetic KB-AT-001 SEQ ID NO 20
QVQLQQSGGDLAQRGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 21 - protein, 9 aa, Artificial
  Synthetic KB-AT-002 CDR1 SEQ ID NO 21
SGRTFNNNG

SEQ ID NO: 22 - protein, 8 aa, Artificial
  Synthetic KB-AT-002 CDR2 SEQ ID NO 22
ISWSGGST

SEQ ID NO: 23 - protein, 17 aa, Artificial
  Synthetic KB-AT-002 CDR3 SEQ ID NO 23
AARTRYNSGLFSRNYDY

SEQ ID NO: 24 - protein, 124 aa, Artificial
  Synthetic KB-AT-002 SEQ ID NO 24
QVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 25 - protein, 9 aa, Artificial
  Synthetic KB-AT-003 CDR1 SEQ ID NO 25
ALTFSSRAW

SEQ ID NO: 26 - protein, 8 aa, Artificial
  Synthetic KB-AT-003 CDR2 SEQ ID NO 26
ITGGGTTN

SEQ ID NO: 27 - protein, 8 aa, Artificial
  Synthetic KB-AT-003 CDR3 SEQ ID NO 27
NGYRYTYA

SEQ ID NO: 28 - protein, 114 aa, Artificial
  Synthetic KB-AT-003 SEQ ID NO 28
QVQLVQSGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSS

SEQ ID NO: 29 - protein, 7 aa, Artificial
  Synthetic KB-AT-004  CDR1 SEQ ID NO 29
AMTFSIR

SEQ ID NO: 30 - protein, 7 aa, Artificial
  Synthetic KB-AT-004 CDR2 SEQ ID NO 30
IGTGDIT

SEQ ID NO: 31 - protein, 8 aa, Artificial
  Synthetic KB-AT-004  CDR3 SEQ ID NO 31
NGYRSTYA

SEQ ID NO: 32 - protein, 113 aa, Artificial
  Synthetic KB-AT-004 SEQ ID NO 32
VQLQQSGGGLVQPGGSLRLSCAASAMTFSIRAWAWYRQAPGKQRELVASIGTGDITNYADSVKGRFTISRDNAKNTFYLQMNSLKPEDTAVYYCNGYRSTYAWGQGTQVTVSS

SEQ ID NO: 33 - protein, 9 aa, Artificial
  Synthetic KB-AT-005 CDR1 SEQ ID NO 33
GRDFNDAAL

SEQ ID NO: 34 - protein, 7 aa, Artificial
  Synthetic KB-AT-005 CDR2 SEQ ID NO 34
ITSGGVR

SEQ ID NO: 35 - protein, 16 aa, Artificial
  Synthetic KB-AT-005 CDR3 SEQ ID NO 35
KADSFKGDYDTSWYLY

SEQ ID NO: 36 - protein, 122 aa, Artificial
  Synthetic KB-AT-005 SEQ ID NO 36
EVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 37 - protein, 8 aa, Artificial
  Synthetic KB-AT-006  CDR1 SEQ ID NO 37
GRTFSNNG

SEQ ID NO: 38 - protein, 8 aa, Artificial
  Synthetic KB-AT-006 CDR2 SEQ ID NO 38
ISWSSGST

SEQ ID NO: 39 - protein, 17 aa, Artificial
  Synthetic KB-AT-006 CDR3 SEQ ID NO 39
AARTRYNSGYFTRNYDY

SEQ ID NO: 40 - protein, 124 aa, Artificial
  Synthetic KB-AT-006 SEQ ID NO 40
QVQLQQSGGGLVQAGGSLRLSCAASGRTFSNNGMGWFRQAPGKEREFVAAISWSSGSTYYADSVKGRYTISRDNAKNTVYLQMNSLKPEDTAVYYCAARTRYNSGYFTRNYDYWGQGTQVTVSS

SEQ ID NO: 41 - protein, 8 aa, Artificial
  Synthetic KB-AT-007 CDR1 SEQ ID NO 41
GRTFRNYV

SEQ ID NO: 42 - protein, 8 aa, Artificial
  Synthetic KB-AT-007 CDR2 SEQ ID NO 42
INRSGAIT

SEQ ID NO: 43 - protein, 16 aa, Artificial
  Synthetic KB-AT-007 CDR3 SEQ ID NO 43
AAGETTWSIRRDDYDY

SEQ ID NO: 44 - protein, 123 aa, Artificial
  Synthetic KB-AT-007 SEQ ID NO 44
QVQLQQSGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSS

SEQ ID NO: 45 - protein, 254 aa, Artificial
  Synthetic KB-AT-002/003 SEQ ID NO 45
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSS

SEQ ID NO: 46 - protein, 263 aa, Artificial
  Synthetic KB-AT-001/002 SEQ ID NO 46
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 47 - protein, 256 aa, Artificial
  Synthetic KB-AT-001/003 SEQ ID NO 47
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 48 - protein, 261 aa, Artificial
  Synthetic KB-AT-001/005 SEQ ID NO 48
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSEVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 49 - protein, 292 aa, Artificial
  Synthetic KB AT-002/003_ABP SEQ ID NO 49
QVQLQESGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTVSSGRGGGSGGGSGGGSGGGSQGLIGDIALPRWGALWGDSV

SEQ ID NO: 50 - protein, 402 aa, Artificial
  Synthetic KB-AT-112 SEQ ID NO 50
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSS

SEQ ID NO: 51 - protein, 395 aa, Artificial
  Synthetic KB-AT-113 SEQ ID NO 51
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 52 - protein, 400 aa, Artificial
  Synthetic KB-AT-115 SEQ ID NO 52
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSEVQLVESGGGLVQPGGSLRLSCEASGRDFNDAALGWSRQVPGKARETVAMITSGGVRNYAETVKDRFTISRDNAKNTVYLDMNNLQPDDTGVYYCKADSFKGDYDTSWYLYWGQGTQVTVSS

SEQ ID NO: 53 - protein, 535 aa, Artificial
  Synthetic KB-AT-1123 SEQ ID NO 53
QVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLQESGGGLVQAGGSLRLSCAASGRTFRNYVMGWFRQAPGKDPEFIAGINRSGAITYYGDSVKGRFTISRDNAKNTVSLQMNSLEPEDTAVYYCAAGETTWSIRRDDYDYWGQGTQVTVSSGGGSGGGSGGGSGGGSQVQLVQSGGGLVQAGGSLRLSCAASGRTFNNNGMGWFRQAPGKEREFVAAISWSGGSTYYADSVKGRYIMSRDNAKNTVYLQMNSLKPEDTAVYYCAARTRYNSGLFSRNYDYWGQGTQVTVSSGGGSGGGSGGGSGGGSQVQLQESGGGLVQPGGSLRLSCAASALTFSSRAWAWYRQAPGKQRELVASITGGGTTNYADSVKGRFTISRDNAKNTVYLQMNSLKPEDTAVHYCNGYRYTYAWGQGTQVTQVTVSS

SEQ ID NO: 54 - protein, 10 aa, Artificial
  Synthetic KB-FIBR-008 CDR1 SEQ ID NO 54
GGSMTSGYAA

SEQ ID NO: 55 - protein, 14 aa, Artificial
  Synthetic KB-FIBR-008  CDR2 SEQ ID NO  55
AYDGSTYYSPSLES

SEQ ID NO: 56 - protein, 8 aa, Artificial
  Synthetic KB-FIBR-008 CDR3 SEQ ID NO  56
RLYNGFEH

SEQ ID NO: 57 - protein, 117 aa, Artificial
  Synthetic KB-FIBR-008 SEQ ID NO  57
QVQLQESGPGLVKPSQTLSLTCTVSGGSMTSGYAAWNWIRQPPGKGLEWMGIIAYDGSTYYSPSLESRTSISRDTSKNQFSLQLSSVTPEDTAVYYCARLYNGFEHWGQGTQVTVSS

SEQ ID NO: 58 - protein, 302 aa, Artificial; identical to SEQ ID NO: 16
  Synthetic KB-FIBR-008bv-ABP SEQ ID NO 58

SEQ ID NO: 59 - protein, 10 aa, Artificial
  Synthetic KB-FIBR-009 CDR1 SEQ ID NO 59
GFTFDDYAMS

SEQ ID NO: 60 - protein, 16 aa, Artificial
  Synthetic KB-FIBR-009 CDR2 SEQ ID NO 60
ISWNGKTTLYAESMKG

SEQ ID NO: 61 - protein, 8 aa, Artificial
  Synthetic KB-FIBR-009 CDR3 SEQ ID NO 61
RGSGLFVS

SEQ ID NO: 62 - protein, 115 aa, Artificial
  Synthetic KB-FIBR-009 SEQ ID NO 62
QLVESGGGLVQPGGSLRLSCAASGFTFDDYAMSWVRQAPGKGLEWVSAISWNGKTTLYAESMKGRFTISRDNAKNTLYLQMNNLKPEDTAVYSCADRGSGLFVSWGQGTQVTVSS

SEQ ID NO: 63 - protein, 284 aa, Artificial
  Synthetic KB-FIBR-009bv-ABP SEQ ID NO 63
QLVESGGGLVQPGGSLRLSCAASGFTFDDYAMSWVRQAPGKGLEWVSAISWNGKTTLYAESMKGRFTISRDNAKNTLYLQMNNLKPEDTAVYSCADRGSGLFVSWGQGTQVTVSSGGGSGGGSGGGSGGGSQLVESGGGLVQPGGSLRLSCAASGFTFDDYAMSWVRQAPGKGLEWVSAISWNGKTTLYAESMKGRFTISRDNAKNTLYLQMNNLKPEDTAVYSCADRGSGLFVSWGQGTQVTVSSGRGGGSLTPRGVRLGGGSQGLIGDIALPRWGALWGDSV

SEQ ID NO: 64 - protein, 10 aa, Artificial
  Synthetic KB-FIBR-011 CDR1 SEQ ID NO 64
GFTFGSYDMN

SEQ ID NO: 65 - protein, 13 aa, Artificial
  Synthetic KB-FIBR-011 CDR2 SEQ ID NO 65
ISYSTWYADSMEG

SEQ ID NO: 66 - protein, 12 aa, Artificial
  Synthetic KB-FIBR-011 CDR3 SEQ ID NO 66
DLVGLVGLEGGY

SEQ ID NO: 67 - protein, 118 aa, Artificial
  Synthetic KB-FIBR-011SEQ ID NO 67
EVQLVESGGGLVQPGGSLRLSCAASGFTFGSYDMNWVRQAPGKGPEWVSTISYSTWYADSMEGRFTISRDNAKNTLYLQMTSLKPEDTAVYYCTTDLVGLVGLEGGYWGQGTQVTVSS

SEQ ID NO: 68 - protein, 290 aa, Artificial
  Synthetic KB-FIBR-011bv_ABP SEQ ID NO 68
EVQLVESGGGLVQPGGSLRLSCAASGFTFGSYDMNWVRQAPGKGPEWVSTISYSTWYADSMEGRFTISRDNAKNTLYLQMTSLKPEDTAVYYCTTDLVGLVGLEGGYWGQGTQVTVSSGGGSGGGSGGGSGGGSEVQLVESGGGLVQPGGSLRLSCAASGFTFGSYDMNWVRQAPGKGPEWVSTISYSTWYADSMEGRFTISRDNAKNTLYLQMTSLKPEDTAVYYCTTDLVGLVGLEGGYWGQGTQVTVSSGRGGGSLTPRGVRLGGGSQGLIGDIALPRWGALWGDSV

SEQ ID NO: 69 - protein, 10 aa, Artificial
  Synthetic KB-FIBR-022  CDR1 SEQ ID NO 69
GFTFDDYAMS

SEQ ID NO: 70 - protein, 16 aa, Artificial
  Synthetic KB-FIBR-022 CDR2 SEQ ID NO 70
INSGGGSTSYADSVKG

SEQ ID NO: 71 - protein, 8 aa, Artificial
  Synthetic KB-FIBR-022 CDR3 SEQ ID NO 71
KIWTQFGY

SEQ ID NO: 72 - protein, 117 aa, Artificial
  Synthetic KB-FIBR-022 SEQ ID NO 72
QVQLVQSGGGLVQPGGSLRLSCAASGFTFDDYAMSWVRQAPGKGLEWVSAINSGGGSTSYADSVKGRFTISRDNDKKTVYLQMNSLKPEDTAVYYCAVKIWTQFGYWGQGTQVTVSS

SEQ ID NO: 73 - protein, 288 aa, Artificial
  Synthetic KB-FIBR-022bv_ABP SEQ ID NO 73
QVQLVQSGGGLVQPGGSLRLSCAASGFTFDDYAMSWVRQAPGKGLEWVSAINSGGGSTSYADSVKGRFTISRDNDKKTVYLQMNSLKPEDTAVYYCAVKIWTQFGYWGQGTQVTVSSGGGSGGGSGGGSGGGSQVQLVQSGGGLVQPGGSLRLSCAASGFTFDDYAMSWVRQAPGKGLEWVSAINSGGGSTSYADSVKGRFTISRDNDKKTVYLQMNSLKPEDTAVYYCAVKIWTQFGYWGQGTQVTVSSGRGGGSLTPRGVRLGGGSQGLIGDIALPRWGALWGDSV

SEQ ID NO: 74 - protein, 10 aa, Artificial
  Synthetic KB-FIBR-048  CDR1 SEQ ID NO 74
KGIFAFNGMG

SEQ ID NO: 75 - protein, 16 aa, Artificial
  Synthetic KB-FIBR-048 CDR2 SEQ ID NO 75
IMQPNGRTIYKDTVKG

SEQ ID NO: 76 - protein, 9 aa, Artificial
  Synthetic KB-FIBR-048 CDR3 SEQ ID NO 76
WVNRTNDMY

SEQ ID NO: 77 - protein, 117 aa, Artificial
  Synthetic KB-FIBR-048 SEQ ID NO 77
QVQLVQSGGGSVQPGGSLRLSCAASKGIFAFNGMGWFRQAPGKERELVAKMQPNGRTIYKDTVKGRFTISRDIQKATVDLLMKSLQPEDTADYYCGAWVNRTNDMYWGQGTQVTVSS

SEQ ID NO: 78 - protein, 288 aa, Artificial
  Synthetic KB-FIBR-048bv_ABP SEQ ID NO 78
QVQLVQSGGGSVQPGGSLRLSCAASKGIFAFNGMGWFRQAPGKERELVAKMQPNGRTIYKDTVKGRFTISRDIQKATVDLLMKSLQPEDTADYYCGAWVNRTNDMYWGQGTQVTVSSGGGSGGGSGGGSGGGSQVQLVQSGGGSVQPGGSLRLSCAASKGIFAFNGMGWFRQAPGKERELVAKMQPNGRTIYKDTVKGRFTISRDIQKATVDLLMKSLQPEDTADYYCGAWVNRTNDMYWGQGTQVTVSSGRGGGSLTPRGVRLGGGSQGLIGDIALPRWGALWGDSV

SEQ ID NO: 79 - protein, 10 aa, Artificial
  Synthetic KB-PAI1-002 CDR1 SEQ ID NO 79
MSSFGMYHMG

SEQ ID NO: 80 - protein, 15 aa, Artificial
  Synthetic KB-PAI1-002 CDR2 SEQ ID NO 80
IGPSGNTYYLDSVKG

SEQ ID NO: 81 - protein, 11 aa, Artificial
  Synthetic KB-PAI1-002 CDR3 SEQ ID NO 81
SRKVVSLAVDY

SEQ ID NO: 82 - protein, 119 aa, Artificial
  Synthetic KB-PAI1-002 SEQ ID NO 82
EVQLVESGGGLVQTGGSLRLSCVASMSSFGMYHMGWYRQAPGSQRKMVAWIGPSGNTYYLDSVKGRFTISRDNAKNTLYLQMNSLKPEDTAVYYCAKSRKVVSLAVDYRGQGTQVTVSS

SEQ ID NO: 83 - protein, 292 aa, Artificial
  Synthetic KB-PAI1-002bv_ABP SEQ ID NO 83
EVQLVESGGGLVQTGGSLRLSCVASMSSFGMYHMGWYRQAPGSQRKMVAWIGPSGNTYYLDSVKGRFTISRDNAKNTLYLQMNSLKPEDTAVYYCAKSRKVVSLAVDYRGQGTQVTVSSGGGSGGGSGGGSGGGSEVQLVESGGGLVQTGGSLRLSCVASMSSFGMYHMGWYRQAPGSQRKMVAWIGPSGNTYYLDSVKGRFTISRDNAKNTLYLQMNSLKPEDTAVYYCAKSRKVVSLAVDYRGQGTQVTVSSGRGGGSLTPRGVRLGGGSQGLIGDIALPRWGALWGDSV

SEQ ID NO: 84 - protein, 10 aa, Artificial
  Synthetic KB-PAI1-006 CDR1 SEQ ID NO 84
GDRLSAYAMG

SEQ ID NO: 85 - protein, 16 aa, Artificial
  Synthetic KB-PAI1-006 CDR2 SEQ ID NO 85
ISRTSGRTYYAGSVKG

SEQ ID NO: 86 - protein, 15 aa, Artificial
  Synthetic KB-PAI1-006 CDR3 SEQ ID NO 86
RYGRYDVARMSRVDY

SEQ ID NO: 87 - protein, 124 aa, Artificial
  Synthetic KB-PAI1-006 SEQ ID NO 87
QVQLVQSGGGLVQAGESLRLSCAASGDRLSAYAMGWFRQGPGKDREFVSAISRTSGRTYYAGSVKGRFTISRDNAKNTVYLQMNSLKAEDTAVYYCAARYGRYDVARMSRVDYWGQGTQVTVSS

SEQ ID NO: 88 - protein, 302 aa, Artificial
  Synthetic KB-PAI1-006bv_ABP SEQ ID NO 88
QVQLVQSGGGLVQAGESLRLSCAASGDRLSAYAMGWFRQGPGKDREFVSAISRTSGRTYYAGSVKGRFTISRDNAKNTVYLQMNSLKAEDTAVYYCAARYGRYDVARMSRVDYWGQGTQVTVSSGGGSGGGSGGGSGGGSQVQLVQSGGGLVQAGESLRLSCAASGDRLSAYAMGWFRQGPGKDREFVSAISRTSGRTYYAGSVKGRFTISRDNAKNTVYLQMNSLKAEDTAVYYCAARYGRYDVARMSRVDYWGQGTQVTVSSGRGGGSLTPRGVRLGGGSQGLIGDIALPRWGALWGDSV

SEQ ID NO: 89 - protein, 10 aa, Artificial
  Synthetic KB-PAI1-007  CDR1 SEQ ID NO  89
GFTFSSHWMN

SEQ ID NO: 90 - protein, 16 aa, Artificial
  Synthetic KB-PAI1-007 CDR2 SEQ ID NO 90
ISGGGFTTYYADFVKG

SEQ ID NO: 91 - protein, 8 aa, Artificial
  Synthetic KB-PAI1-007 CDR3 SEQ ID NO 91
GPWQDVDA

SEQ ID NO: 92 - protein, 117 aa, Artificial
  Synthetic KB-PAI1-007 SEQ ID NO 92
EVQLVQSGGGLVQPGGSLRLSCAASGFTFSSHWMNWVRQAPGKGLQWISTISGGGFTTYYADFVKGRFTISRDNAKNTLYLQMNSLKSEDTAVYYCAKGPWQDVDAWGQGTQVTVSS

SEQ ID NO: 93 - protein, 288 aa, Artificial
  Synthetic KB-PAI1-007bv_ABP SEQ ID NO 93
EVQLVQSGGGLVQPGGSLRLSCAASGFTFSSHWMNWVRQAPGKGLQWISTISGGGFTTYYADFVKGRFTISRDNAKNTLYLQMNSLKSEDTAVYYCAKGPWQDVDAWGQGTQVTVSSGGGSGGGSGGGSGGGSEVQLVQSGGGLVQPGGSLRLSCAASGFTFSSHWMNWVRQAPGKGLQWISTISGGGFTTYYADFVKGRFTISRDNAKNTLYLQMNSLKSEDTAVYYCAKGPWQDVDAWGQGTQVTVSSGRGGGSLTPRGVRLGGGSQGLIGDIALPRWGALWGDSV

SEQ ID NO: 94 - protein, 8 aa, Artificial
  Synthetic thrombin-cleavage site SEQ ID NO 94
LTPRGVRL